(19) United States
(12) Patent Application Publication
(10) Pub. No.: US 2024/0075139 A1
(43) Pub. Date: Mar. 7, 2024

(54) Antibodies against infectious pathogens and method for production thereof

(71) Applicants: Universität zu Köln, Köln (DE); Yeda Research and Development Co., Ltd. Weizmann Institute of Science, Rehovot (IL)

(72) Inventors: Florian Klein, Köln (DE); Matthias Zehner, Bonn (DE); Stefanie Ehrhardt, Köln (DE); Ron Diskin, Rehovot (IL)

(21) Appl. No.: 17/766,705

(22) PCT Filed: Oct. 7, 2020
(86) PCT No.: PCT/EP2020/078109

§ 371 Date: Apr. 5, 2022

(30) Foreign Application Priority Data
Oct. 7, 2019 (EP) 19201736.6

(51) Int. Cl. A61K 39/42; A61P 37/04

(52) U.S. Cl. CPC A61K 39/42; A61P 37/04; A61K 2039/55

(57) ABSTRACT

The present invention relates to a method for obtaining monoclonal human antibodies directed against an infectious pathogen, monoclonal human antibodies or binding fragments thereof which are directed against an infectious pathogen as obtained by the method of the invention, a pharmaceutical composition comprising such monoclonal human antibodies, a kit comprising such antibodies, and the monoclonal antibodies or binding fragments thereof and the pharmaceutical composition and the kit for use as a medicament and in the treatment or prevention of a disease caused by the infectious pathogen.

### TECHNICAL FIELD

The present invention relates to a method for obtaining monoclonal human antibodies directed against an infectious pathogen, monoclonal human antibodies or binding fragments thereof which are directed against an infectious pathogen as obtained by the method of the invention, a pharmaceutical composition comprising such monoclonal human antibodies, a kit comprising such antibodies, and the monoclonal human antibodies or binding fragments thereof, the pharmaceutical composition and the kit for use as a medicament and for use in the treatment or prevention of a disease caused by the infectious pathogen.

### BACKGROUND OF THE INVENTION

Ebolaviruses can cause severe infections in humans leading to unpredictable epidemics with mortality rates up to 90%. The devastating outbreak in West Africa (2013-2016) caused >11,000 confirmed deaths. The severe and since 2018 ongoing outbreak in the Democratic Republic of the Congo (DRC) highlights the significant impact of Ebola virus disease (EVD) and the critical need for efficacious countermeasures.

Different Ebolavirus species can cause symptomatic infection in humans. These are Sudan Ebolavirus (SUDV), Bundibugyo Ebolavirus (BDBV), and Zaire Ebolavirus (EBOV), of which the latter accounts for the 2018-2019 outbreak in the DRC. Studies in animal models demonstrated that neutralizing antibodies can prevent infection and are effective for post-exposure prophylaxis.

Therefore, monoclonal antibodies have been evaluated in clinical trials and administered to patients suffering from EVD. For instance, a combination of three antibodies obtained from immunized mice (REGN-EB3) was tested in a phase I clinical trial (Sivapalasingam, S. et al. The Lancet Infectious Diseases 18, 884-893, doi:10.1016/s1473-3099(18)30397-9 (2018). Moreover, the neutralizing antibody mAb114 that was isolated from an EVD survivor has been tested in a clinical trial and demonstrated a preferential pharmacokinetic and safety profile (Gaudinski, M. R. et al. The Lancet, doi:10.1016/s0140-6736(19)30036-4 (2019).

Until recently, efficacy data in humans were mostly limited to the ZMapp cocktail comprising three human-mouse chimeric antibodies called 13C6, C2G4, and C4G7. During the 2013-2016 epidemic, ZMapp was tested in EVD patients, reducing fatality rates from 37% to 22% (Davey, R. T., Jr. et al. N Engl J Med 375, 1448-1456, doi:10.1056/NEJMoa1604330 (2016).

More promising results were very recently reported for REGN-EB3 and mAb114 that were administered to EVD patients in a multicenter, randomized controlled clinical trial. In an interim analysis from 499 participants, lethal outcome was markedly reduced from 67% to 29% and 34% for REGN-EB3 and mAb114, respectively (Kupferschmidt, K. Science Latest News, doi:10.1126/science.aaz1032 (2019).

In addition to passive immunotherapy, several vaccine candidates have been developed. Among them, the recombinant vesicular stomatitis virus (VSV)-based vector carrying the EBOV glycoprotein (rVSV-ZEBOV) is the most advanced vaccine and has been administered to >180,000 individuals. rVSV-ZEBOV has been demonstrated to be protective against lethal EBOV challenges in rodents and NHPs.

Moreover, results of two ring vaccination trials estimate vaccine efficiency to be around 97%. As a vaccine candidate with efficacy, rVSV-ZEBOV has been designated as a lead candidate for administration in current and future EVD outbreaks. However, despite its broad application, a detailed understanding of the rVSV-ZEBOV immune response is still limited and no analysis has been performed to elucidate the molecular composition of the induced antibody response.

To date, only a very limited number of functional antibodies against infectious pathogens such as the Ebola virus are available of which only a very small percentage is monoclonal and of a human origin, thus having excellent expected tolerability when administered to human patients.

Additional monoclonal antibodies of a human origin against evasive and highly infectious pathogens, such as viruses including Ebola virus, HI virus and others, are difficult to obtain and highly sought after. Such antibodies could allow a treatment of infected patients, prevention of large outbreaks or contraction of the disease by subjects at risk and could thus serve to additionally reduce the high mortality rate observed in EVD patients and other patients suffering from or at risk or contracting infectious diseases associated with a high mortality.

Prior investigations have exclusively focused on extracting potentially functional and specific antibodies against infectious pathogens from surviving patients, as is the case for known and established functional human Ebola antibodies.

This, however, poses the problem of an extremely limited availability of antibody sources in form of rare survivors of the diseases caused by infectious pathogens. The cases of mAb100 and mAb114 which are accepted for administration either in combination or as mAb114 monotherapy for EVD patients exemplify the difficulty of obtaining additional and novel antibodies from human survivors.

Both of mAb100 and mAb114 have been recovered from the same, single individual after over twenty years after recovery from Ebola infection. This alone already shows the difficulties and time which may be required after infection to successfully obtain additional functional antibodies from disease survivors as antibody sources.

Most of all, the strategy of obtaining antibodies from human survivors has two other significant disadvantages.

First, it must be possible for humans to survive an infection and/or develop immunity against the pathogen to be able to serve as potential sources for such antibodies. This can be extremely difficult for various diseases including inter alia Ebola, Marburg, Rabies or Lassa viruses but not limited thereto. This means that survivors can be extremely difficult or even impossible to find as useful sources for human antibodies for certain diseases.

Second, aiming at survivors of a disease caused by an infectious pathogen as a source for functional human antibodies necessarily requires human subjects, which have contracted that disease and suffer or have suffered from the diseases' symptoms. This also means that an endemic or an epidemic involving numerous sick or dying patients has to necessarily precede the option of obtaining new treatment possibilities.

Both disadvantages are sought to be overcome in the search for novel human monoclonal antibodies directed against infectious pathogens, such as Ebola virus.

Thus, it is an object of the present invention to provide a novel method for obtaining additional human monoclonal antibodies against infectious pathogens, which does not require finding patients having survived the disease caused by such infectious pathogens. Furthermore, it is another object of the present invention to provide novel human monoclonal antibodies against such diseases caused by infectious pathogens.

### SUMMARY OF THE INVENTION

These objects have been solved by the aspects of the present invention as specified hereinafter.

According to the first aspect of the present invention, a method for obtaining human antibodies directed against an infectious pathogen, preferably against a virus, is provided, wherein the method comprises vaccination of a human subject with a vaccine comprising an antigenic protein derived from the infectious pathogen, preferably an antigenic protein ordinarily expressed on the surface of the infectious pathogen, isolating B lymphocytes exhibiting antibodies directed against the infectious pathogen on their cell surface and/or antibodies directed against the infectious pathogen from vaccinated subjects, identifying B lymphocytes expressing antibodies directed against the infectious pathogen and/or antibodies directed against the infectious pathogen, preferably on a single cell- or single antibody-basis, and expressing monoclonal antibodies directed against the infectious pathogen.

According to a preferred embodiment of the first aspect of the present invention, the vaccination is an intentional vaccination of a human subject.

According to another preferred embodiment of the first aspect of the present invention, the infectious pathogen is a virus from the family Filoviridae, more preferably from the genus Ebolavirus or Marburgvirus, even more preferably from the genus Ebolavirus, and/or wherein the antigenic protein derived from the infectious pathogen is an Ebolavirus glycoprotein.

According to yet another preferred embodiment of the first aspect of the present invention, the antigenic protein is present in the vaccine as part of an attenuated virus, which is different from the infectious pathogen, more preferably as part of an attenuated recombinant vesicular stomatitis virus.

According to a specifically preferred embodiment of the first aspect of the present invention, the vaccine comprises rVSV-ZEBOV.

According to one preferred embodiment of the first aspect of the present invention, the step of isolating B lymphocytes exhibiting antibodies directed against the infectious pathogen on their cell surface and/or antibodies directed against the infectious pathogen comprises first extracting B lymphocytes of the vaccinated subject, and then carrying out serial limiting dilutions, more preferably to single cell level.

According to another preferred embodiment of the first aspect of the present invention, the step of isolating B lymphocytes exhibiting antibodies directed against the infectious pathogen on their cell surface and/or antibodies directed against the infectious pathogen comprises first extracting B lymphocytes of the vaccinated subject and then contacting the extracted B lymphocytes with a component of the infectious pathogen to allow selective sorting of the extracted B lymphocytes which exhibit antibodies directed against the infectious pathogen on their cell surface, more preferably wherein selective sorting comprises fluorescence-activated cell sorting (FACS), magnetic-activated cell sorting (MACS) or chromatographic separation.

According to a more preferred embodiment of the previous preferred embodiment of the first aspect of the present invention, B lymphocytes exhibiting antibodies directed against the infectious pathogen on their cell surface and/or antibodies directed against the infectious pathogen are isolated from vaccinated subjects by contacting extracted B lymphocytes with a component of the infectious pathogen labelled with a fluorescence marker, and isolating B lymphocytes exhibiting antibodies directed against the infectious pathogen on their cell surface by fluorescence-activated cell sorting (FACS), even more preferably in a single cell-manner.

According to a preferred embodiment of the first aspect of the present invention, the step of identifying B lymphocytes exhibiting antibodies directed against the infectious pathogen on their cell surface and/or antibodies directed against the infectious pathogen, more preferably on a single cell- or single antibody-basis, comprises amplifying nucleotide sequences coding for light and heavy chains of each individual antibody, and sequencing the amplified nucleotide sequences of said light and heavy chains.

According to another preferred embodiment of the first aspect of the present invention, the step of expressing a monoclonal antibody directed against the infectious pathogen comprises cloning of the light and heavy chain sequences into a mAb expression vector, transfecting the mAb expression vector into a suitable host cell, culturing the suitable host cell, and harvesting the expressed monoclonal antibody from the host cell supernatant.

According to the second aspect of the present invention, a monoclonal human antibody or binding fragment thereof is provided which is directed against an infectious pathogen, wherein the human antibody is obtained by the method according to the first aspect of the present invention.

According to a preferred embodiment of the second aspect of the present invention, the infectious pathogen is a virus from the family Filoviridae, more preferably from the genus Ebolavirus or Marburgvirus, even more preferably from the genus Ebolavirus.

According to another preferred embodiment of the second aspect of the present invention, the monoclonal human antibody comprises an amino acid sequence encoded by the heavy chain V gene segment IGHV3-15 or an amino acid sequence being at least 80% identical thereto, and an amino acid sequence encoded by the light chain V gene segment IGLV1-40 or an amino acid sequence being at least 80% identical thereto.

According to one preferred embodiment of the second aspect of the present invention, the monoclonal human antibody or binding fragment thereof exhibits reactivity with more than one virus from the family Filoviridae, more preferably with at least two selected from the group comprising Bundibugyo virus, Marburg virus, Sudan virus, and Zaire Ebolavirus.

According to another preferred embodiment of the second aspect of the present invention, the monoclonal human antibody or binding fragment thereof exhibits neutralization of Ebolavirus in a neutralization assay at a concentration of 1 μg/mL or less, preferably at 0.74 μg/mL or less, more preferably at 0.05 μg/mL or less, even more preferably at 0.01 μg/mL or less.

According to one preferred embodiment of the second aspect of the present invention, the monoclonal human antibody or binding fragment thereof exhibits superior neutralization of Ebolavirus in a neutralization assay in comparison to mAb100.

According to a preferred embodiment of the second aspect of the present invention, the antibody or binding fragment thereof comprises the heavy chain CDR1 to CDR3 and the light chain CDR1 to CDR3 amino acid sequence of one antibody from the group comprising 1T0129 (consisting of the heavy chain amino acid sequence of SEQ ID No. 1 and the light chain amino acid sequence of SEQ ID No. 2), 1T0139 (consisting of the heavy chain amino acid sequence of SEQ ID No. 3 and the light chain amino acid sequence of SEQ ID No. 4), 1T0201 (consisting of the heavy chain amino acid sequence of SEQ ID No. 5 and the light chain amino acid sequence of SEQ ID No. 6), 1T0221 (consisting of the heavy chain amino acid sequence of SEQ ID No. 7 and the light chain amino acid sequence of SEQ ID No. 8), 1T0225 (consisting of the heavy chain amino acid sequence of SEQ ID No. 9 and the light chain amino acid sequence of SEQ ID No. 10), 1T0227 (consisting of the heavy chain amino acid sequence of SEQ ID No. 11 and the light chain amino acid sequence of SEQ ID No. 12), 1T0248 (consisting of the heavy chain amino acid sequence of SEQ ID No. 13 and the light chain amino acid sequence of SEQ ID No. 14), 1T0321 (consisting of the heavy chain amino acid sequence of SEQ ID No. 15 and the light chain amino acid sequence of SEQ ID No. 16), 1T0325 (consisting of the heavy chain amino acid sequence of SEQ ID No. 17 and the light chain amino acid sequence of SEQ ID No. 18), 1T0371 (consisting of the heavy chain amino acid sequence of SEQ ID No. 19 and the light chain amino acid sequence of SEQ ID No. 20), 1T0451 (consisting of the heavy chain amino acid sequence of SEQ ID No. 21 and the light chain amino acid sequence of SEQ ID No. 22), 1T0455 (consisting of the heavy chain amino acid sequence of SEQ ID No. 23 and the light chain amino acid sequence of SEQ ID No. 24), 1T0465 (consisting of the heavy chain amino acid sequence of SEQ ID No. 25 and the light chain amino acid sequence of SEQ ID No. 26), 1T0473 (consisting of the heavy chain amino acid sequence of SEQ ID No. 27 and the light chain amino acid sequence of SEQ ID No. 28), 1T0552 (consisting of the heavy chain amino acid sequence of SEQ ID No. 29 and the light chain amino acid sequence of SEQ ID No. 30), 1T0655 (consisting of the heavy chain amino acid sequence of SEQ ID No. 31 and the light chain amino acid sequence of SEQ ID No. 32), 3T0123 (consisting of the heavy chain amino acid sequence of SEQ ID No. 33 and the light chain amino acid sequence of SEQ ID No. 34), 3T0202 (consisting of the heavy chain amino acid sequence of SEQ ID No. 35 and the light chain amino acid sequence of SEQ ID No. 36), 3T0215 (consisting of the heavy chain amino acid sequence of SEQ ID No. 37 and the light chain amino acid sequence of SEQ ID No. 38), 3T0245 (consisting of the heavy chain amino acid sequence of SEQ ID No. 39 and the light chain amino acid sequence of SEQ ID No. 40), 3T0253 (consisting of the heavy chain amino acid sequence of SEQ ID No. 41 and the light chain amino acid sequence of SEQ ID No. 42), 3T0258 (consisting of the heavy chain amino acid sequence of SEQ ID No. 43 and the light chain amino acid sequence of SEQ ID No. 44), 3T0265 (consisting of the heavy chain amino acid sequence of SEQ ID No. 45 and the light chain amino acid sequence of SEQ ID No. 46), 3T0331 (consisting of the heavy chain amino acid sequence of SEQ ID No. 47 and the light chain amino acid sequence of SEQ ID No. 48), 3T0338 (consisting of the heavy chain amino acid sequence of SEQ ID No. 49 and the light chain amino acid sequence of SEQ ID No. 50), 3T0350 (consisting of the heavy chain amino acid sequence of SEQ ID No. 51 and the light chain amino acid sequence of SEQ ID No. 52), 3T0405 (consisting of the heavy chain amino acid sequence of SEQ ID No. 53 and the light chain amino acid sequence of SEQ ID No. 54), 3T0415 (consisting of the heavy chain amino acid sequence of SEQ ID No. 55 and the light chain amino acid sequence of SEQ ID No. 56), 3T0468 (consisting of the heavy chain amino acid sequence of SEQ ID No. 57 and the light chain amino acid sequence of SEQ ID No. 58), 3T0478 (consisting of the heavy chain amino acid sequence of SEQ ID No. 59 and the light chain amino acid sequence of SEQ ID No. 60), 3T0553 (consisting of the heavy chain amino acid sequence of SEQ ID No. 61 and the light chain amino acid sequence of SEQ ID No. 62), 3T0611 (consisting of the heavy chain amino acid sequence of SEQ ID No. 63 and the light chain amino acid sequence of SEQ ID No. 64), 3T0650 (consisting of the heavy chain amino acid sequence of SEQ ID No. 65 and the light chain amino acid sequence of SEQ ID No. 66), 3T0673 (consisting of the heavy chain amino acid sequence of SEQ ID No. 67 and the light chain amino acid sequence of SEQ ID No. 68), 4m0333 (consisting of the heavy chain amino acid sequence of SEQ ID No. 69 and the light chain amino acid sequence of SEQ ID No. 70), 4m0368 (consisting of the heavy chain amino acid sequence of SEQ ID No. 71 and the light chain amino acid sequence of SEQ ID No. 72), 4m0373 (consisting of the heavy chain amino acid sequence of SEQ ID No. 73 and the light chain amino acid sequence of SEQ ID No. 74), 4T0115 (consisting of the heavy chain amino acid sequence of SEQ ID No. 75 and the light chain amino acid sequence of SEQ ID No. 76), 4T0154 (consisting of the heavy chain amino acid sequence of SEQ ID No. 77 and the light chain amino acid sequence of SEQ ID No. 78), 4T0159 (consisting of the heavy chain amino acid sequence of SEQ ID No. 79 and the light chain amino acid sequence of SEQ ID No. 80), 4T0176 (consisting of the heavy chain amino acid sequence of SEQ ID No. 81 and the light chain amino acid sequence of SEQ ID No. 82), 4T0182 (consisting of the heavy chain amino acid sequence of SEQ ID No. 83 and the light chain amino acid sequence of SEQ ID No. 84), 4T0243 (consisting of the heavy chain amino acid sequence of SEQ ID No. 85 and the light chain amino acid sequence of SEQ ID No. 86), 4T0262 (consisting of the heavy chain amino acid sequence of SEQ ID No. 87 and the light chain amino acid sequence of SEQ ID No. 88), 4T0284 (consisting of the heavy chain amino acid sequence of SEQ ID No. 89 and the light chain amino acid sequence of SEQ ID No. 90), 4T0306 (consisting of the heavy chain amino acid sequence of SEQ ID No. 91 and the light chain amino acid sequence of SEQ ID No. 92), 4T0344 (consisting of the heavy chain amino acid sequence of SEQ ID No. 93 and the light chain amino acid sequence of SEQ ID No. 94), 4T0365 (consisting of the heavy chain amino acid sequence of SEQ ID No. 95 and the light chain amino acid sequence of SEQ ID No. 96), 4T0412 (consisting of the heavy chain amino acid sequence of SEQ ID No. 97 and the light chain amino acid sequence of SEQ ID No. 98), 4T0423 (consisting of the heavy chain amino acid sequence of SEQ ID No. 99 and the light chain amino acid sequence of SEQ ID No. 100), 4T0427 (consisting of the heavy chain amino acid sequence of SEQ ID No. 101 and the light chain amino acid sequence of SEQ ID No. 102), 4T0429 (consisting of the heavy chain amino acid sequence of SEQ ID No. 103 and the light chain amino acid sequence of SEQ ID No. 104), 4T0434 (consisting of the heavy chain amino acid sequence of SEQ ID No. 105 and the light chain amino acid sequence of SEQ ID No. 106), 4T0444 (consisting of the heavy chain amino acid sequence of SEQ ID No. 107 and the light chain amino acid sequence of SEQ ID No. 108), 4T0452 (consisting of the heavy chain amino acid sequence of SEQ ID No. 109 and the light chain amino acid sequence of SEQ ID No. 110), 4T0525 (consisting of the heavy chain amino acid sequence of SEQ ID No. 111 and the light chain amino acid sequence of SEQ ID No. 112), 4T0541 (consisting of the heavy chain amino acid sequence of SEQ ID No. 113 and the light chain amino acid sequence of SEQ ID No. 114), 4T0570 (consisting of the heavy chain amino acid sequence of SEQ ID No. 115 and the light chain amino acid sequence of SEQ ID No. 116), 4T0578 (consisting of the heavy chain amino acid sequence of SEQ ID No. 117 and the light chain amino acid sequence of SEQ ID No. 118), 4T0657 (consisting of the heavy chain amino acid sequence of SEQ ID No. 119 and the light chain amino acid sequence of SEQ ID No. 120), 4T0726 (consisting of the heavy chain amino acid sequence of SEQ ID No. 121 and the light chain amino acid sequence of SEQ ID No. 122), 4T0764 (consisting of the heavy chain amino acid sequence of SEQ ID No. 123 and the light chain amino acid sequence of SEQ ID No. 124), 4T0784 (consisting of the heavy chain amino acid sequence of SEQ ID No. 125 and the light chain amino acid sequence of SEQ ID No. 126), 5T0107 (consisting of the heavy chain amino acid sequence of SEQ ID No. 127 and the light chain amino acid sequence of SEQ ID No. 128), 5T0180 (consisting of the heavy chain amino acid sequence of SEQ ID No. 129 and the light chain amino acid sequence of SEQ ID No. 130), 5T0202 (consisting of the heavy chain amino acid sequence of SEQ ID No. 131 and the light chain amino acid sequence of SEQ ID No. 132), 5T0209 (consisting of the heavy chain amino acid sequence of SEQ ID No. 133 and the light chain amino acid sequence of SEQ ID No. 134), 5T0223 (consisting of the heavy chain amino acid sequence of SEQ ID No. 135 and the light chain amino acid sequence of SEQ ID No. 136), 5T0257 (consisting of the heavy chain amino acid sequence of SEQ ID No. 137 and the light chain amino acid sequence of SEQ ID No. 138), 5T0278 (consisting of the heavy chain amino acid sequence of SEQ ID No. 139 and the light chain amino acid sequence of SEQ ID No. 140), 5T0337 (consisting of the heavy chain amino acid sequence of SEQ ID No. 141 and the light chain amino acid sequence of SEQ ID No. 142), 5T0376 (consisting of the heavy chain amino acid sequence of SEQ ID No. 143 and the light chain amino acid sequence of SEQ ID No. 144), 5T0378 (consisting of the heavy chain amino acid sequence of SEQ ID No. 145 and the light chain amino acid sequence of SEQ ID No. 146), 5T0404 (consisting of the heavy chain amino acid sequence of SEQ ID No. 147 and the light chain amino acid sequence of SEQ ID No. 148), 5T0406 (consisting of the heavy chain amino acid sequence of SEQ ID No. 149 and the light chain amino acid sequence of SEQ ID No. 150), 5T0420 (consisting of the heavy chain amino acid sequence of SEQ ID No. 151 and the light chain 25 amino acid sequence of SEQ ID No. 152), 5T0451 (consisting of the heavy chain amino acid sequence of SEQ ID No. 153 and the light chain amino acid sequence of SEQ ID No. 154), 5T0465 (consisting of the heavy chain amino acid sequence of SEQ ID No. 155 and the light chain amino acid sequence of SEQ ID No. 156), more preferably of one antibody from the group comprising 1T0325, 1T0451, 1T0473, 1T0655, 3T0123, 3T0253, 3T0553, 4T0243, 4T0306, 4T0570, 4T0726, 4T0764, 4T0784, 5T0180, 5T0223, 5T0278, 5T0337, 5T0451.

According to another preferred embodiment of the second aspect of the present invention, the antibody or binding fragment thereof comprises the combination of the heavy chain and the light chain of one antibody selected from the group comprising 1T0129 (SEQ ID No. 1 and 2), 1T0139 (SEQ ID No. 3 and 4), 1T0201 (SEQ ID No. 5 and 6),1T0221 (SEQ ID No. 7 and 8), 1T0225 (SEQ ID No. 9 and 10), 1T0227 (SEQ ID No. 11 and 12), 1T0248 (SEQ ID No. 13 and 14), 1T0321 (SEQ ID No. 15 and 16), 1T0325 (SEQ ID No. 17 and 18), 1T0371 (SEQ ID No. 19 and 20), 1T0451 (SEQ ID No. 21 and 22), 1T0455 (SEQ ID No. 23 and 24), 1T0465 (SEQ ID No. 25 and 26), 1T0473 (SEQ ID No. 27 and 28), 1T0552 (SEQ ID No. 29 and 30), 1T0655 (SEQ ID No. 31 and 32), 3T0123 (SEQ ID No. 33 and 34), 3T0202 (SEQ ID No. 35 and 36), 3T0215 (SEQ ID No. 37 and 38), 3T0245 (SEQ ID No. 39 and 40), 3T0253 (SEQ ID No. 41 and 42), 3T0258 (SEQ ID No. 43 and 44), 3T0265 (SEQ ID No. 45 and 46), 3T0331 (SEQ ID No. 47 and 48), 3T0338 (SEQ ID No. 49 and 50), 3T0350 (SEQ ID No. 51 and 52), 3T0405 (SEQ ID No. 53 and 54), 3T0415 (SEQ ID No. 55 and 56), 3T0468 (SEQ ID No. 57 and 58), 3T0478 (SEQ ID No. 59 and 60), 3T0553 (SEQ ID No. 61 and 62), 3T0611 (SEQ ID No. 63 and 64), 3T0650 (SEQ ID No. 65 and 66), 3T0673 (SEQ ID No. 67 and 68), 4m0333 (SEQ ID No. 69 and 70), 4m0368 (SEQ ID No. 71 and 72), 4m0373 (SEQ ID No. 73 and 74), 4T0115 (SEQ ID No. 75 and 76), 4T0154 (SEQ ID No. 77 and 78), 4T0159 (SEQ ID No. 79 and 80), 4T0176 (SEQ ID No. 81 and 82), 4T0182 (SEQ ID No. 83 and 84), 4T0243 (SEQ ID No. 85 and 86), 4T0262 (SEQ ID No. 87 and 88), 4T0284 (SEQ ID No. 89 and 90), 4T0306 (SEQ ID No. 91 and 92), 4T0344 (SEQ ID No. 93 and 94), 4T0365 (SEQ ID No. 95 and 96), 4T0412 (SEQ ID No. 97 and 98), 4T0423 (SEQ ID No. 99 and 100), 4T0427 (SEQ ID No. 101 and 102), 4T0429 (SEQ ID No. 103 and 104), 4T0434 (SEQ ID No. 105 and 106), 4T0444 (SEQ ID No. 107 and 108), 4T0452 (SEQ ID No. 109 and 110), 4T0525 (SEQ ID No. 111 and 112), 4T0541 (SEQ ID No. 113 and 114), 4T0570 (SEQ ID No. 115 and 116), 4T0578 (SEQ ID No. 117 and 118), 4T0657 (SEQ ID No. 119 and 120), 4T0726 (SEQ ID No. 121 and 122), 4T0764 (SEQ ID No. 123 and 124), 4T0784 (SEQ ID No. 125 and 126), 5T0107 (SEQ ID No. 127 and 128), 5T0180 (SEQ ID No. 129 and 130), 5T0202 (SEQ ID No. 131 and 132), 5T0209 (SEQ ID No. 133 and 134), 5T0223 (SEQ ID No. 135 and 136), 5T0257 (SEQ ID No. 137 and 138), 5T0278 (SEQ ID No. 139 and 140), 5T0337 (SEQ ID No. 141 and 142), 5T0376 (SEQ ID No. 143 and 144), 5T0378 (SEQ ID No. 145 and 146), 5T0404 (SEQ ID No. 147 and 148), 5T0406 (SEQ ID No. 149 and 150), 5T0420 (SEQ ID No. 151 and 152), 5T0451 (SEQ ID No. 153 and 154), 5T0465 (SEQ ID No. 155 and 156), more preferably of one antibody from the group comprising 1T0325, 1T0451, 1T0473, 1T0655, 3T0123, 3T0253, 3T0553, 4T0243, 4T0306, 4T0570, 4T0726, 4T0764, 4T0784, 5T0180, 5T0223, 5T0278, 5T0337, 5T0451.

According to one preferred embodiment of the second aspect of the present invention, the antibody or binding fragment thereof comprises the heavy chain CDR1 to CDR3 and the light chain CDR1 to CDR3 amino acid sequence of one of the antibodies selected from 3T0331 (consisting of the heavy chain amino acid sequence of SEQ ID No. 47 and the light chain amino acid sequence of SEQ ID No. 48) or 4T0243 (consisting of the heavy chain amino acid sequence of SEQ ID No. 85 and the light chain amino acid sequence of SEQ ID No. 86), more preferably of antibody 3T0331.

According to a preferred embodiment of the second aspect of the present invention, the antibody or binding fragment thereof comprises the combination of the heavy chain and the light chain of one of the antibodies selected from 3T0331 (SEQ ID No. 47 and 48) or 4T0243 (SEQ ID No. 85 and 86), more preferably of 3T0331.

According to the third aspect of the present invention, a pharmaceutical composition is provided comprising a monoclonal human antibody or binding fragment thereof according to the second aspect of the present invention, and at least one pharmaceutically acceptable excipient.

According to a preferred embodiment of the third aspect of the present invention, the pharmaceutical composition is a vaccination composition for a human subject.

According to the fourth aspect of the present invention, a kit is provided comprising a monoclonal human antibody or binding fragment thereof according to the second aspect of the present invention, and a container.

According to the fifth aspect of the present invention, a monoclonal human antibody or binding fragment thereof according to the second aspect of the invention, a pharmaceutical composition according to the third aspect of the invention, or a kit according to the fourth aspect of the invention are provided for use as a medicament, preferably for use as a vaccine.

According to the sixth aspect of the present invention, a monoclonal human antibody or binding fragment thereof according to the second aspect of the invention, a pharmaceutical composition according to the third aspect of the invention, or a kit according to the fourth aspect of the invention are provided for use in the treatment or prevention of a disease caused by the infectious pathogen in human subjects, preferably for use in the treatment or prevention of Ebolavirus-caused disease in human subjects.

According to another aspect of the present invention, a method is provided for obtaining human antibodies directed against an infectious pathogen, wherein the method comprises identifying in an isolate taken from a human subject previously vaccinated with a vaccine comprising an antigenic protein derived from the infectious pathogen, wherein the isolate is an isolate of B lymphocytes exhibiting antibodies directed against the infectious pathogen on their cell surface and/or antibodies directed against the infectious pathogen from vaccinated subjects, those B lymphocytes expressing antibodies directed against the infectious pathogen and/or antibodies directed against the infectious pathogen, preferably on a single cell- or single antibody-basis, and expressing monoclonal antibodies directed against the infectious pathogen. According to a preferred embodiment of this aspect, the method is carried out in vitro.

### DESCRIPTION OF FIGURES

FIG. 1 shows a comparison of serum reactivity of survivors and vaccinated individuals. For this serum reactivity to EBOV GPΔTM (Makona) measured by ELISA in rVSV-ZEBOV-immunized subjects (black) is compared to EVD survivors (S1-S7, gray, left panel). Error bars show standard deviation (SD) of means of technical duplicates. Lines indicate means of calculated EC₅₀ dilution factors, which are 35 and 456 for vaccines and survivors, respectively (n=6 [EV] and 7 [S] independent samples, p=0.0013, right panel). PBS and healthy serum samples were used as negative controls. Significance was tested by a two-tailed unpaired t-test.

FIG. 2 shows a schematic overview of the sample collection as well as the schematic structure of the EBOV GP used for B lymphocyte sorting, wherein according to (a) sample collection from rVSV-ZEBOV-immunized donors (EV01-EV06) was performed between 18.5 to 26 months after single dose vaccination. Serum was collected and PBMC samples were obtained after leukapheresis or large blood draw. Part (b) shows the design of EBOV GPΔTM construct with GCN4 trimerization domain, His- and Avi-Tag, as well as covalent labeling with fluorophore DyLight 488 (dark gray).

FIG. 3 shows the gating strategy for flow cytometric analysis and B lymphocyte sorting. CD19-enriched (MACS) PBMCs of rVSV-ZEBOV-vaccinated donors (EV01, EV03-EV05) were gated for lymphocytes −>live cells −>single cells. CD20⁺IgG⁺ B lymphocytes were selected for sort of EBOV-specific B lymphocytes. One of two independent experiments with similar results is shown.

FIG. 4 shows the binding activity and cross-reactivity of antibodies isolated from vaccinated individuals as well as their ability to neutralize EBOV. (a) ELISA binding analysis of mAbs isolated from rVSV-ZEBOV-vaccinated individuals (EV01, EV03-EV05) against EBOV GPΔTM. Shown are EC₅₀ values of binding mAbs [n=21 (EV01), 23 (EV03), 29 (EV04) and 16 (EV05)]. (b) EC₅₀ values [n=42 (BDBV), 32 (SUDV) and 4 (MARV) of EBOV GPΔTM-specific mAbs with cross-reactivity against GPs from different filoviruses determined by ELISA. mAbs specific for GPs of EBOV plus one other filovirus are shown in white or light gray colors and mAbs specific for GPs of EBOV plus 2 or 3 other filovirus species are shown in dark gray or black, respectively. (a-b) Means are indicated by lines. (c) Analysis of serum neutralizing activity of EVD survivors (light gray) and rVSV-ZEBOV-immunized donors (EV; black). Shown are dilution factors yielding full neutralization of EBOV Mayinga. Colored background areas indicate means. Significance was tested by two-tailed unpaired t-test (n=7 (survivor) and n=6 (EV) biologically independent samples, p=0.089). (d) Antibody concentrations (μg/mL) required to achieve full neutralization. mAbs obtained from rVSV-ZEBOV-immunized subjects [dark gray; n=9 (EV01), 14 (EV03), 10 (EV04), 9 (EV05)] compared to published antibodies (light gray; KZ52, mAb114, mAb100, ADI-15758, ADI-15999, and ADI-16037). Background area illustrates neutralization range of reference antibodies. Means are indicated by lines. Neutralization experiments were performed in quadruplicates.

FIG. 5 shows the importance of the monoclonal antibodies with the V gene combination of IGHV3-15 and IGLV1-40 for the EBOV neutralization activity in the vaccinated individuals. (a) Frequency of heavy chain (left) and light chain (right) V genes in rVSV-ZEBOV-induced antibodies that neutralize EBOV Mayinga (dark gray), bind (light gray) or do not bind EBOV GPΔTM (white; n=143). Shown are V genes with at least one neutralizing antibody. Significant changes in V gene frequency of neutralizing antibodies were tested using a binominal test under the null hypothesis of equal frequency, α=0.05, and Bonferroni correction. Only IGHV3-15 and IGLV1-40 increases were highly significant with p=2.2×10⁻⁹. (b) Frequency of neutralizing IGHV3-15-carrying antibodies. Slices represent antibodies of unrelated clones from individual donors (left) and corresponding light chains (right).

### DETAILED DESCRIPTION OF THE INVENTION

The present inventors have dedicated themselves to solving the problem of the present invention and were successful to find a novel method for obtaining human monoclonal antibodies against infectious pathogens, in particular against the Ebola virus, which overcomes the disadvantages of the prior art strategies, and were further successful in providing novel and useful human monoclonal antibodies against the Ebola virus that have been obtained by said method.

When setting out to make the present invention, the present inventors have initially analysed serum samples of subjects vaccinated with a vaccine against Ebola virus. These serum samples of vaccinated patients have then been compared against serum samples of surviving Ebola patients with regard to serum reactivity and binding to the EBOV glycoprotein in ELISAs.

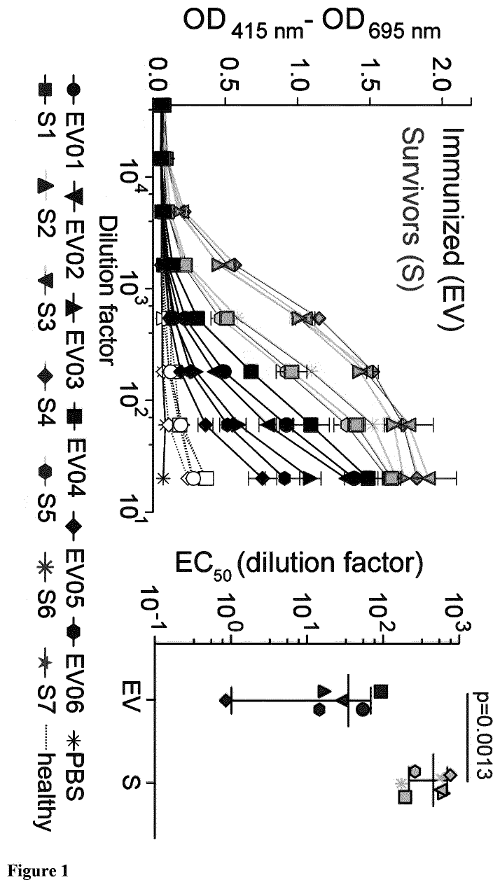

Interestingly, serum samples of vaccinated subjects showed a significantly lower activity in comparison to serum samples of survivors (FIG. 1). This finding initially pointed away from any expectation of success in finding novel and useful monoclonal antibodies directed against Ebola virus in vaccinated individuals and would have pointed to rather employing disease survivors as antibody sources instead.

Nevertheless, the present inventors proceeded with their approach and successfully completed the invention of a novel and useful method for obtaining human monoclonal antibodies against infectious pathogens, in particular against Ebola virus, and of novel and useful monoclonal, human antibodies against such infectious pathogens.

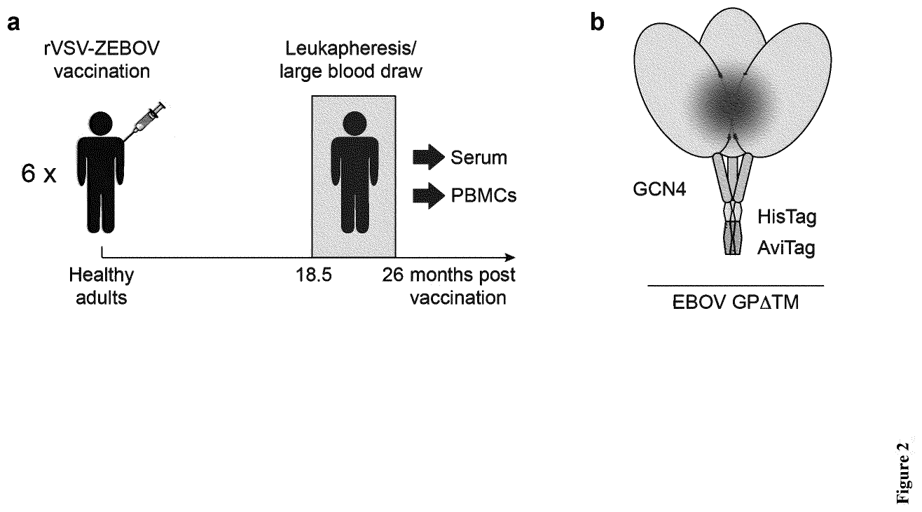

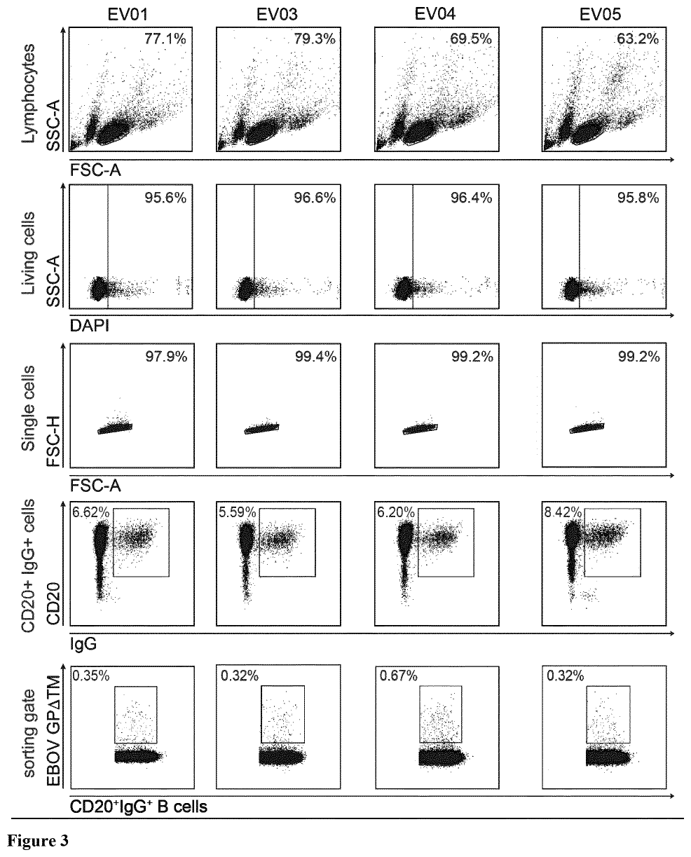

Thus, the present invention provides a method for obtaining human antibodies directed against an infectious pathogen, wherein the method comprises vaccination of a human subject with a vaccine comprising an antigenic protein derived from the infectious pathogen (FIG. 2), preferably an antigenic protein ordinarily expressed on the surface of the infectious pathogen, isolating B lymphocytes exhibiting antibodies directed against the infectious pathogen on their cell surface and/or antibodies directed against the infectious pathogen from vaccinated subjects (FIG. 3), identifying B lymphocytes expressing antibodies directed against the infectious pathogen and/or antibodies directed against the infectious pathogen, preferably on a single cell- or single antibody-basis, and expressing monoclonal antibodies directed against the infectious pathogen.

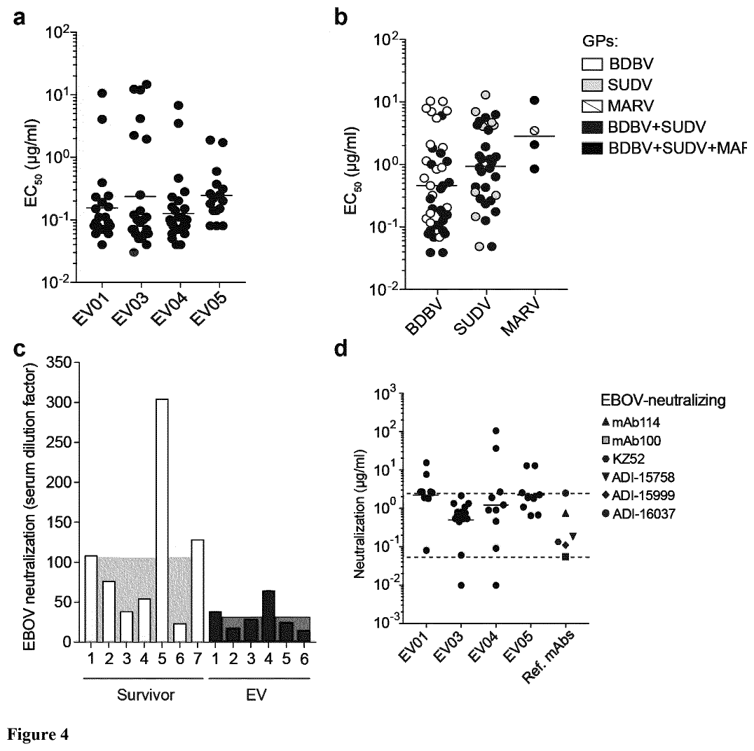

Preferably within the present invention, the antibodies directed against an infectious pathogen are directed against a virus. More preferably, the antibodies are directed against a virus from the family Filoviridae, even more preferably against a virus from the genus Ebolavirus or Marburgvirus, particularly preferably from the genus Ebolavirus (FIG. 4a and b).

According to a preferred embodiment, an antibody directed against an infectious pathogen means an antibody binding to the infectious pathogen, or a protein or proteinaceous component of the infectious pathogen with an at least 10-fold, more preferably at least 50-fold, particularly preferably at least 100-fold increased affinity compared to unrelated infectious pathogens, proteins or proteinaceous components thereof.

According to another preferred embodiment, an antibody directed against an infectious pathogen means an antibody binding to an epitope of the infectious pathogen with an at least 10-fold, more preferably at least 50-fold, particularly preferably more at least 100-fold increased affinity compared to epitopes of unrelated infectious pathogens. Within the context of the above, unrelated infectious pathogens mean infectious pathogens from a different family or higher taxonomic rank.

In a preferred embodiment of the present invention, the vaccination of a human subject is an intentional vaccination. This vaccination may be carried out according to established principles for vaccinating human patients, as for example described in Vaccines (Eds. Plotkin, S. A., et al., 7^{th} Edition, Elsevier Health Sciences Division, 2017, ISBN 978-0-323-35761-6).

The vaccination of a human subject is carried out with a vaccine comprising an antigenic protein derived from the infectious pathogen. Such antigenic protein will serve as an immunogen for the generation of autologous antibodies by the immune system of the vaccinated subject. With the aim of obtaining antibodies, which are most suitable for administration to human patients, production and isolation of human antibodies is clearly advantageous to generation of antibodies in other species. Advantages of such antibodies may include increased longevity, long half-life after being administered to the subject, no or minor side effects and other desirable properties.

According to a preferred embodiment, the antigenic protein derived from the infectious pathogen is ordinarily expressed on the surface of the infectious pathogen. In the context of the present invention, an antigenic protein being ordinarily expressed on the surface of the infectious pathogen means a protein which is expressed in this way when being in its natural environment and part of the infectious pathogen as it occurs in nature.

For the sake of being used for vaccination of a human subject, the antigenic protein may preferably be present in the vaccine as part of an attenuated virus different from the infectious pathogen. In this way, the antigenic protein can be presented to the patient in order to provoke an immune response without the apparent risk of causing unwanted or dangerous disease symptoms. More preferably, the antigenic protein may be part of an attenuated recombinant vesicular stomatitis virus.

According to one preferred embodiment, the antigenic protein derived from the infectious pathogen as used in the method of the invention is an Ebolavirus glycoprotein, more preferably the envelope glycoprotein of the Zaire Ebolavirus (strain Kikwit-95) having the UniProt accession number P87666. Alternatively preferably, proteins of other infectious pathogens may be integrated in a heterologous carrier to be used as vaccines for human subjects.

In the case of viruses as infectious pathogens, it may be preferable to use an envelope glycoprotein of the infectious pathogen and introduce it into a heterologous virus as a replacement for the proprietary envelope glycoprotein of the carrier virus. In particular, it is preferred that an envelope glycoprotein of a virus as the infectious pathogen is used as replacement for the envelope glycoprotein (UniProt accession number P03522) of the attenuated recombinant vesicular stomatitis virus used as carrier virus.

In a particularly preferred embodiment of the present invention, the vaccine comprises rVSV-ZEBOV. This vaccine has previously been used to vaccinate patients at risk of developing EVD symptoms and is described inter alia in Monath, T P et al. Vaccine: X, Volume 1, 2019, 100009, ISSN 2590-1362, https://doi.org/10.1016/j.jvacx.2019.100009 and the references cited therein. To date, more than 180,000 people have been actively immunized using this vaccine and could serve as potential sources for obtaining monoclonal, human antibodies using the method of the present invention.

The method of the present invention further involves a step of isolating B lymphocytes exhibiting antibodies directed against the infectious pathogen on their cell surface from vaccinated subjects and/or isolating antibodies directed against the infectious pathogen from vaccinated subjects (FIG. 3). Preferably, this step is a step of isolating B lymphocytes exhibiting antibodies directed against the infectious pathogen on their cell surface from vaccinated subjects. More preferably, this step involves collecting peripheral blood mononuclear cells (PBMC) from vaccinated individuals as an initial separation step.

According to a preferred embodiment, peripheral blood mononuclear cells (PBMC) are enriched for B lymphocytes, preferably by enrichment of B lymphocytes via magnetic cell separation (MACS), more preferably by use of magnetic cell separation (MACS) using CD19-microbeads. According to another preferred embodiment, B lymphocytes of the vaccinated subject are subjected to serial limiting dilutions which is followed by identification of B lymphocytes expressing antibodies directed against the infectious pathogen. Preferably, serial limiting dilutions are carried out to statistically obtain not more than three cells per well upon plating, more preferably not more than two cells per well upon plating, particularly preferably a single cell per well upon plating. Also preferably, a step of identifying the B lymphocytes obtained or the antibodies secreted by said lymphocytes is carried out following the serial limiting dilutions.

According to one preferred embodiment, the step of isolating B lymphocytes exhibiting antibodies directed against the infectious pathogen on their cell surface comprises contacting the extracted B lymphocytes with a component of the infectious pathogen to allow selective sorting of the B lymphocytes of interest from the extracted B lymphocytes. Preferably, selective sorting of the B lymphocytes of interest comprises fluorescence-activated cell sorting (FACS), magnetic-activated cell sorting (MACS) or chromatographic separation.

According to a preferred embodiment of the present invention, the step of isolating B lymphocytes may further comprise contacting the cells with additional markers to allow selective sorting of CD20⁺ and/or IgG⁺ cells (FIG. 3). According to one preferred embodiment, B lymphocytes exhibiting antibodies directed against the infectious pathogen on their cell surface are isolated from vaccinated subjects by contacting the extracted B lymphocytes with a component of the infectious pathogen labelled with a fluorescence marker, and isolating the B lymphocytes of interest by fluorescence-activated cell sorting (FACS) (FIG. 3).

Preferably therein, the B lymphocytes are contacted with components of the infectious pathogen ordinarily expressed on the surface of the infectious pathogen, more preferably envelope glycoproteins of viruses lacking the transmembrane domain. In preferable embodiments of the invention relating to Ebolavirus, the B lymphocytes are contacted with Ebolavirus glycoprotein lacking the transmembrane domain, more preferably with recombinant Ebolavirus GP constructs lacking the transmembrane domain residues 651-676.

According to a particularly preferred embodiment, the B lymphocytes are contacted with a protein of SEQ ID No. 158 (peptide sequence of EBOV GPΔTM), alternatively preferably by a protein encoded by SEQ ID No. 157 (nucleotide sequence of EBOV GPΔTM). The construct EBOV GPΔTM which displays the envelope protein of Ebola virus for binding to antibodies specific for said protein is able to selectively associate with antibodies directed against Ebola virus. Since EBOV GPΔTM further comprises a fluorescent marker, the step of contacting allows selective marking of specific antibodies for fluorescence-activated cell sorting (FACS).

Within the context of the present invention, it is also well possible to use other components of infectious pathogens which are bound by specific antibodies extracted from vaccinated human subjects in a selective manner. Also, other markers aside from the fluorescent marker employed in EBOV GPΔTM are also encompassed by the present invention to be fused to components of infectious pathogens, be it other fluorescent markers or specifically binding molecules which allow specific sorting by other methods.

According to a preferred embodiment of the present invention, the step of identifying B lymphocytes exhibiting antibodies directed against the infectious pathogen on their cell surface and/or antibodies directed against the infectious pathogen comprises amplifying nucleotide sequences coding for light and heavy chains of each individual antibody and sequencing the amplified nucleotide sequences of said light and heavy chains.

According to one preferred embodiment, the step of identifying B lymphocytes of interest comprises carrying out single cell PCR, more preferably using sequential, semi-nested PCRs. Single cell PCR may preferably be carried out as previously described by Tiller, T. et al. Journal of Immunological Methods, 329, 112-124 (2008). or by von Boehmer, L. et al. Nat Protoc 11, 1908-1923 (2016).

Preferably, after the nucleotide sequences coding for the light and heavy chains have been sequenced and identified, a step of expressing each monoclonal antibody directed against the infectious pathogen is carried out which comprises cloning of the light and heavy chain nucleotide sequences into a mAb expression vector, more preferably using sequence- and ligation-independent cloning (SLIC) as previously described by by von Boehmer, L. et al. Nat Protoc 11, 1908-1923 (2016).

Furthermore, the resulting plasmids are preferably transfected into suitable host cells, more preferably into HEK293E cells. Transfected cells are then preferably cultured in selective media to select for successfully transfected cells. The supernatant of said cells are preferably collected and antibodies were isolated from the supernatant, preferably by using protein G sepharose.

According to the present invention, the step of isolating B lymphocytes exhibiting antibodies directed against the infectious pathogen on their cell surface may also be understood as isolating a population of B lymphocytes including such B lymphocytes from the vaccinated subject. Further preferably, the method for obtaining human antibodies according to the present invention should be carried out by performing steps a), b), c) and d) in this order.

According to the the second aspect of the present invention, a monoclonal human antibody or binding fragment thereof is provided which is directed against an infectious pathogen, wherein the human antibody is preferably obtained by the method according to the first aspect of the present invention.

As already laid out above, monoclonal antibodies of human origin are difficult to obtain by using the standard immunological toolkit. Antibody sources for the production of monoclonal antibodies are limited and isolation and identification of specifically binding antibodies which neutralize Ebola virus are challenging. Therefore, it would have been a very high burden for the skilled person to identify and generate additional antibodies able to neutralize Ebola virus.

The present inventors however succeeded in this task and herewith provide novel antibodies which neutralize Ebola virus and other infectious pathogens with unexpected properties. These antibodies have been obtained by the method of the present invention.

Within the context of the present invention, the antibodies, which have been generated and described herein, may be used and claimed as the complete monoclonal human antibody or as any functional or binding fragment thereof. Preferably, the monoclonal human antibody or any kind of functional or binding fragment thereof should comprise the complementarity determining regions (CDR) 1 to 3 of the heavy chain and of the light chain of the human monoclonal antibody.

The CDR regions of the antibody sequences described herein are preferably defined according to the numbering scheme of IMGT which is an adaptation of the numbering scheme of Chothia (ImMunoGeneTics information system®; Lefranc et al., NAR 27: 209-212 (1999); http://imgt.cines.fr).

In one preferred embodiment, the antibody is a monoclonal antibody or a fragment thereof that retains binding specificity and ability to neutralize infectious pathogen. In one preferred embodiment, the antibody is an IgG1, IgG2, IgG3, or IgG4 antibody. For example, the antibody may be an antibody comprising an Fc domain of any human IgG isotype (e.g. IgG1, IgG2, IgG3, or IgG4).

Optionally, the antigen-binding compound consists of or comprises a Fab, Fab′, Fab′-SH, F(ab)₂, Fv, a diabody, single-chain antibody fragment, or a multispecific antibody comprising multiple different antibody fragments.

According to a preferred embodiment, the monoclonal antibody or binding fragment thereof directed against an infectious pathogen is directed against a virus from the family Filoviridae, more preferably from the genus Ebolavirus or Marburgvirus, particularly preferably from the genus Ebolavirus (FIG. 4a and b).

According to one preferred embodiment, the monoclonal human antibody or binding fragment thereof exhibits cross-reactivity against a broad range of infectious pathogens, more preferably against a range of infectious pathogens within the same species, even more preferably within the same genus, particularly preferably within the same family (FIG. 4b).

According to one specific preferred embodiment, the monoclonal human antibody or binding fragment thereof exhibits reactivity with more than one virus from the family Filoviridae, preferably with at least two selected from the group comprising Bundibugyo virus, Marburg virus, Sudan virus, and Zaire Ebolavirus. Antibodies which exhibit such reactivity are antibodies 1T0129, 1T0221, 1T0225, 1T0321, 1T0325, 1T0371, 1T0451, 1T0465, 1T0473, 1T0552, 1T0655, 3T0123, 3T0215, 3T0253, 3T0350, 3T0405, 3T0415, 3T0553, 4m0333, 4m0373, 4T0115, 4T0154, 4T0159, 4T0176, 4T0182, 4T0243, 4T0262, 4T0284, 4T0306, 4T0344, 4T0365, 4T0412, 4T0423, 4T0427, 4T0434, 4T0525, 4T0541, 4T0570, 4T0657, 4T0726, 4T0764, 4T0784, 5T0107, 5T0180, 5T0223, 5T0257, 5T0278, 5T0337, 5T0378, 5T0404, 5T0406, 5T0420 and 5T0451.

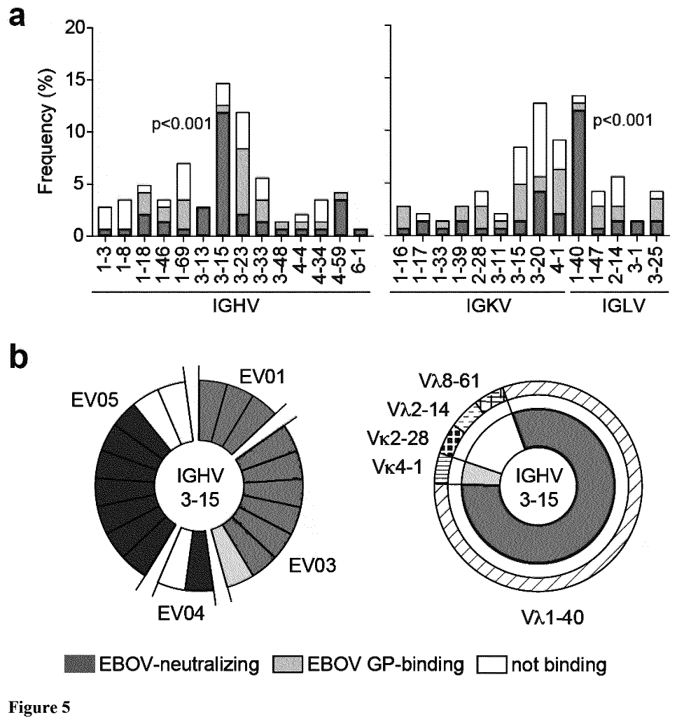

According to another preferred embodiment, the monoclonal human antibody comprises an amino acid sequence encoded by the heavy chain V gene segment IGHV3-15 or an amino acid sequence being at least 80% identical thereto, and an amino acid sequence encoded by the light chain V gene segment IGLV1-40 or an amino acid sequence being at least 80% identical thereto (FIG. 5). Antibodies, which are exhibiting such a distinct combination of V gene segments and successfully neutralize Ebola virus are 1T0201, 1T0227, 1T0455, 3T0245, 3T0253, 3T0265, 3T0338, 3T0650, 3T0673, 4T0452, 5T0180, 5T0202, 5T0209, 5T0223, 5T0278, 5T0337 and 5T0451.

It was surprising to find that a high percentage of the antibodies which were identified as potent neutralizers to the Ebola virus possess the combination of the heavy chain V gene segment IGHV3-15 and the light chain V gene segment IGLV1-40 (FIG. 5). It appears as if this combination alone may be suitable to act as Ebola virus neutralizing antibody. Small variations and mutations of the sequence of the antibody may be tolerated without negatively influencing the activity of the claimed antibodies. Thus, amino acid sequences of the antibodies described herein are considered to be encompassed by the present invention as long as they exhibit an identity ratio of 80% to the disclosed sequences.

The determination of percent identity between two sequences is accomplished according to the present invention by using the mathematical algorithm of Karlin and Altschul (Proc. Natl. Acad. Sci. USA (1993) 90: 5873-5877). Such an algorithm is the basis of the BLASTN and BLASTP programs of Altschul et al. (J. Mol. Biol. (1990) 215: 403-410). BLAST nucleotide searches are performed with the BLASTN program. To obtain gapped alignments for comparative purposes, Gapped BLAST is utilized as described by Altschul et al. (Nucleic Acids Res. (1997) 25: 3389-3402). When utilizing BLAST and Gapped BLAST programs, the default parameters of the respective programs are used.

According to a preferred embodiment of the present invention, amino acid sequences form part of the invention which consist of or comprise a nucleic acid sequence being at least 85% identical to the individualized aptamer sequences which are disclosed herein, more preferably at least 90% identical, even more preferred at least 95% identical.

According to another preferred embodiment, the monoclonal human antibody or binding fragment thereof exhibits neutralization of Ebolavirus in a neutralization assay at a concentration of 15 μg/mL or less, preferably at 5 μg/mL or less, more preferably at 0.1 μg/mL or less, even more preferably at 0.05 μg/mL or less (FIG. 4c and d).

The neutralization assay to be used for determining the values given above is carried out as follows:

Antibodies (starting at a concentration of 100 μg/ml [antibodies]) were serially diluted in 96-well culture plates in Dulbecco's Modified Eagle's Medium (DMEM, Thermo Fisher Scientific) supplemented with 2% fetal calf serum, penicillin (50 units/m1), streptomycin (50 μg/ml) and L-glutamine (2 mM).

100 TCID₅₀ units of EBOV Mayinga (GenBank: NC_002549) and SUDV Boniface (GenBank: FJ968794.1) were added to the serum or antibody dilutions. Following incubation at 37° C. for 1 hour, Vero C1008 cells (ATCC® CRL-1586™; 10,000 cells per well) were added. Plates were then incubated at 37° C. with 5% CO₂ and cytopathic effects (CPE) were evaluated at day 7 post infection as previously reported in Cutts et al. Scientific Reports (2019) 9:6590 | https://doi.org/10.1038/s41598-019-42386-5.

Neutralization may be defined as visibly recognizable reduction of CPE in serum or antibody dilutions compared to positive controls. Neutralization titers were calculated of four replicates as geometric mean concentrations in the case of monoclonal antibodies (GMC). The cut-off of the assay is determined by the first dilution of the respective serum or antibody.

According to one preferred embodiment, the concentrations of antibody required for neutralization mean the concentrations used on 100 TCID₅₀ units of a virus which prevents development of cytopathic effects of the virus on human or NHP cells. According to a specific embodiment, side-by-side comparison of the neutralization assay may be carried out with positive and negative controls to determine neutralization.

Antibodies which exhibit neutralization at a concentration of more than 15 μg/mL in the neutralization assay described above are 1T0130 and 1T0221, antibodies which exhibit neutralization at a concentration of 15 μg/mL or less and more than 5 μg/mL are 1T0139, 5T0180, 5T0376 and 1T0248, antibodies which exhibit neutralization at a concentration of 5 μg/mL or less and more than 0.1 μg/mL are 4m0368, 3T0265, 4T0578, 3T0123, 3T0202, 3T0673, 3T0245, 5T0278, 5T0465, 3T0650, 3T0478, 3T0611, 4T0452, 4T0764, 5T0451, 3T0338, 4T0444, 3T0253, 3T0553, 1T0455, 5T0209, 1T0227, 4T0784, 5T0223, 3T0468, 5T0337, 1T0451, 5T0202, 4T0726, 1T0201, 1T0325 and 1T0473, antibodies which exhibit neutralization at a concentration of 0.1 μg/mL or less and more than 0.05 μg/mL are 3T0258, 1T0655, 4T0570, antibodies which exhibit neutralization at a concentration of 0.05 μg/mL or less are 3T0331 and 4T0243.

As examined in the neutralization assay, it could be experimentally verified that the following antibodies show neutralization at the following concentrations: 3T0331 (0.011 μg/mL), 4T0243 (0.011 μg/mL), 3T0258 (0.06 μg/mL), 1T0655 (0.08 μg/mL), 4T0570 (0.09 μg/mL), 4m0368 (0.13 μg/mL), 3T0265 (0.44 μg/mL), 4T0578 (0.45 μg/mL), 3T0123 (0.52 μg/mL), 3T0202 (0.53 μg/mL), 3T0673 (0.53 μg/mL), 3T0245 (0.63 μg/mL), 5T0278 (0.63 μg/mL), 5T0465 (0.66 μg/mL), 3T0650 (0.74 μg/mL), 3T0478 (0.78 μg/mL), 3T0611 (0.78 μg/mL), 4T0452 (0.88 μg/mL), 4T0764 (0.88 μg/mL), 5T0451 (1.05 μg/mL), 3T0338 (1.06 μg/mL), 4T0444 (1.2 μg/mL), 3T0253 (1.31 μg/m L), 3T0553 (1.31 μg/mL), 1T0455 (1.77 μg/mL), 5T0209 (1.77 μg/mL), 1T0227 (1.86 μg/mL), 4T0784 (1.86 μg/mL), 5T0223 (1.86 μg/mL), 3T0468 (2.1 μg/mL), 5T0337 (2.21 μg/mL), 1T0451 (2.5 μg/mL), 5T0202 (2.5 μg/mL), 4T0726 (2.56 μg/mL), 1T0201 (2.63 μg/mL), 1T0325 (2.63 μg/mL), 1T0473 (2.63 μg/mL), 1T0139 (7.44 μg/mL), 5T0180 (12,5 μg/mL), 5T0376 (12.5 μg/mL), 1T0248 (14.8 μg/mL), 4T0429 (35 μg/mL), 4T0306 (100 μg/mL).

As a reference value, it may be mentioned that the antibody mAb114, which is presently administered to Ebola patients, exhibits neutralization at a concentration of 0.744 μg/mL (FIG. 4d).

According to a preferred embodiment of the present invention, the monoclonal human antibody or binding fragment thereof is or may be derived from one antibody from the group comprising 1T0325, 1T0451, 1T0473, 1T0655, 3T0123, 3T0253, 3T0553, 4T0243, 4T0306, 4T0570, 4T0726, 4T0764, 4T0784, 5T0180, 5T0223, 5T0278, 5T0337, 5T0451. More preferably the monoclonal human antibody or binding fragment thereof is or may be derived from the antibody 4T0243 or 3T0331, even more preferably from the antibody 3T0331.

In the description of the present application, antibody designations may be used. It is pointed out that the antibodies consist of heavy and light chains which also form part of the present description. If reference is made to an antibody by its designation or to a SEQ ID No., it should be understood that these ways of reference are interchangeable.

In this regard, the following antibodies under their designation are herewith disclosed to be made up of the following heavy and light chains:
1T0129 (consisting of the heavy chain amino acid sequence of SEQ ID No. 1 and the light chain amino acid sequence of SEQ ID No. 2),
1T0139 (heavy chain SEQ ID No. 3 and the light chain SEQ ID No. 4),
1T0201 (heavy chain SEQ ID No. 5 and the light chain SEQ ID No. 6),
1T0221 (heavy chain SEQ ID No. 7 and the light chain SEQ ID No. 8),
1T0225 (heavy chain SEQ ID No. 9 and the light chain SEQ ID No. 10),
1T0227 (heavy chain SEQ ID No. 11 and the light chain SEQ ID No. 12),
1T0248 (heavy chain SEQ ID No. 13 and the light chain SEQ ID No. 14),
1T0321 (heavy chain SEQ ID No. 15 and the light chain SEQ ID No. 16),
1T0325 (heavy chain SEQ ID No. 17 and the light chain SEQ ID No. 18),
1T0371 (heavy chain SEQ ID No. 19 and the light chain SEQ ID No. 20),
1T0451 (heavy chain SEQ ID No. 21 and the light chain SEQ ID No. 22),
1T0455 (heavy chain SEQ ID No. 23 and the light chain SEQ ID No. 24),
1T0465 (heavy chain SEQ ID No. 25 and the light chain SEQ ID No. 26),
1T0473 (heavy chain SEQ ID No. 27 and the light chain SEQ ID No. 28),
1T0552 (heavy chain SEQ ID No. 29 and the light chain SEQ ID No. 30),
1T0655 (heavy chain SEQ ID No. 31 and the light chain SEQ ID No. 32),
3T0123 (heavy chain SEQ ID No. 33 and the light chain SEQ ID No. 34),
3T0202 (heavy chain SEQ ID No. 35 and the light chain SEQ ID No. 36),
3T0215 (heavy chain SEQ ID No. 37 and the light chain SEQ ID No. 38),
3T0245 (heavy chain SEQ ID No. 39 and the light chain SEQ ID No. 40),
3T0253 (heavy chain SEQ ID No. 41 and the light chain SEQ ID No. 42),
3T0258 (heavy chain SEQ ID No. 43 and the light chain SEQ ID No. 44),
3T0265 (heavy chain SEQ ID No. 45 and the light chain SEQ ID No. 46),
3T0331 (heavy chain SEQ ID No. 47 and the light chain SEQ ID No. 48),
3T0338 (heavy chain SEQ ID No. 49 and the light chain SEQ ID No. 50),
3T0350 (heavy chain SEQ ID No. 51 and the light chain SEQ ID No. 52),
3T0405 (heavy chain SEQ ID No. 53 and the light chain SEQ ID No. 54),
3T0415 (heavy chain SEQ ID No. 55 and the light chain SEQ ID No. 56),
3T0468 (heavy chain SEQ ID No. 57 and the light chain SEQ ID No. 58),
3T0478 (heavy chain SEQ ID No. 59 and the light chain SEQ ID No. 60),
3T0553 (heavy chain SEQ ID No. 61 and the light chain SEQ ID No. 62),
3T0611 (heavy chain SEQ ID No. 63 and the light chain SEQ ID No. 64),
3T0650 (heavy chain SEQ ID No. 65 and the light chain SEQ ID No. 66),
3T0673 (heavy chain SEQ ID No. 67 and the light chain SEQ ID No. 68),
4m0333 (heavy chain SEQ ID No. 69 and the light chain SEQ ID No. 70),
4m0368 (heavy chain SEQ ID No. 71 and the light chain SEQ ID No. 72),
4m0373 (heavy chain SEQ ID No. 73 and the light chain SEQ ID No. 74),
4T0115 (heavy chain SEQ ID No. 75 and the light chain SEQ ID No. 76),
4T0154 (heavy chain SEQ ID No. 77 and the light chain SEQ ID No. 78),
4T0159 (heavy chain SEQ ID No. 79 and the light chain SEQ ID No. 80),
4T0176 (heavy chain SEQ ID No. 81 and the light chain SEQ ID No. 82),
4T0182 (heavy chain SEQ ID No. 83 and the light chain SEQ ID No. 84),
4T0243 (heavy chain SEQ ID No. 85 and the light chain SEQ ID No. 86),
4T0262 (heavy chain SEQ ID No. 87 and the light chain SEQ ID No. 88),
4T0284 (heavy chain SEQ ID No. 89 and the light chain SEQ ID No. 90),
4T0306 (heavy chain SEQ ID No. 91 and the light chain SEQ ID No. 92),
4T0344 (heavy chain SEQ ID No. 93 and the light chain SEQ ID No. 94),
4T0365 (heavy chain SEQ ID No. 95 and the light chain SEQ ID No. 96),
4T0412 (heavy chain SEQ ID No. 97 and the light chain SEQ ID No. 98),
4T0423 (heavy chain SEQ ID No. 99 and the light chain SEQ ID No. 100),
4T0427 (heavy chain SEQ ID No. 101 and the light chain SEQ ID No. 102),
4T0429 (heavy chain SEQ ID No. 103 and the light chain SEQ ID No. 104),
4T0434 (heavy chain SEQ ID No. 105 and the light chain SEQ ID No. 106),
4T0444 (heavy chain SEQ ID No. 107 and the light chain SEQ ID No. 108),
4T0452 (heavy chain SEQ ID No. 109 and the light chain SEQ ID No. 110),
4T0525 (heavy chain SEQ ID No. 111 and the light chain SEQ ID No. 112),
4T0541 (heavy chain SEQ ID No. 113 and the light chain SEQ ID No. 114),
4T0570 (heavy chain SEQ ID No. 115 and the light chain SEQ ID No. 116),
4T0578 (heavy chain SEQ ID No. 117 and the light chain SEQ ID No. 118),
4T0657 (heavy chain SEQ ID No. 119 and the light chain SEQ ID No. 120),
4T0726 (heavy chain SEQ ID No. 121 and the light chain SEQ ID No. 122),
4T0764 (heavy chain SEQ ID No. 123 and the light chain SEQ ID No. 124),
4T0784 (heavy chain SEQ ID No. 125 and the light chain SEQ ID No. 126),
5T0107 (heavy chain SEQ ID No. 127 and the light chain SEQ ID No. 128),
5T0180 (heavy chain SEQ ID No. 129 and the light chain SEQ ID No. 130),
5T0202 (heavy chain SEQ ID No. 131 and the light chain SEQ ID No. 132),
5T0209 (heavy chain SEQ ID No. 133 and the light chain SEQ ID No. 134),
5T0223 (heavy chain SEQ ID No. 135 and the light chain SEQ ID No. 136),
5T0257 (heavy chain SEQ ID No. 137 and the light chain SEQ ID No. 138),
5T0278 (heavy chain SEQ ID No. 139 and the light chain SEQ ID No. 140),
5T0337 (heavy chain SEQ ID No. 141 and the light chain SEQ ID No. 142),
5T0376 (heavy chain SEQ ID No. 143 and the light chain SEQ ID No. 144),
5T0378 (heavy chain SEQ ID No. 145 and the light chain SEQ ID No. 146),
5T0404 (heavy chain SEQ ID No. 147 and the light chain SEQ ID No. 148),
5T0406 (heavy chain SEQ ID No. 149 and the light chain SEQ ID No. 150),
5T0420 (heavy chain SEQ ID No. 151 and the light chain SEQ ID No. 152),
5T0451 (heavy chain SEQ ID No. 153 and the light chain SEQ ID No. 154),
5T0465 (heavy chain SEQ ID No. 155 and the light chain SEQ ID No. 156)

The present invention further relates to a pharmaceutical composition comprising a monoclonal human antibody or binding fragment thereof according to the invention as defined and further described herein and at least one pharmaceutically acceptable excipient. Preferably, the pharmaceutical composition is a vaccination composition for a human subject.

The present invention also encompasses a kit comprising a monoclonal human antibody or binding fragment thereof according to the invention as defined and further described herein and a container.

In one aspect, the present invention is also directed to the monoclonal human antibody or binding fragment thereof according to the invention as defined and further described herein, the pharmaceutical composition as described herein and the kit for use as a medicament, preferably for use as a vaccine.

In another aspect, the present invention is also directed to the monoclonal human antibody or binding fragment thereof according to the invention as defined and further described wherein, the pharmaceutical composition as described herein and the kit for use in the treatment or prevention of a disease caused by the infectious pathogen in a human subject, preferably for use in the treatment or prevention of Ebolavirus-caused disease in human subjects.

In one other aspect, the present invention is also directed to a method of treatment of a patient suffering from a disease caused by an infectious pathogen, preferably Ebolavirus-caused disease, wherein the patient is administered an effective amount of the monoclonal human antibody or binding fragment thereof according to the invention or a pharmaceutical composition of the invention.

In another aspect, the present invention is also directed to the use of the monoclonal human antibody or binding fragment thereof according to the invention or a pharmaceutical composition of the invention in the manufacture of a medicament for treatment of a disease caused by an infectious pathogen, preferably Ebolavirus-caused disease.

All embodiments of the present invention as described herein are deemed to be combinable in any combination, unless the skilled person considers such a combination to not make any technical sense.

### EXAMPLES

rVSV-ZEBOV-GP-Vaccinated Individuals and Sample Collection

Individuals participating in this study were previously enrolled in the Phase I Trial to Assess the Safety, Tolerability and Immunogenicity of an Ebola Virus Vaccine (rVSV4G-ZEBOV-GP, NCT02283099) and were vaccinated with either 3×10⁵ or 3×10⁶ plaque forming units (pfu).

From this cohort, four individuals were enrolled in an observational study (INA; 16-054) at the University Hospital of Cologne to collect PBMC and serum samples. The protocol was approved by the Institutional Review Board (IRB) of the University of Cologne, Germany. From all participants, serum or plasma samples, and EDTA blood or leukapheresis samples were collected and peripheral blood mononuclear cells (PBMC) were purified from EDTA blood or leukapheresis by density gradient centrifugation using HistoPaque and stored in FBS containing 10% DMSO at −150° C. Serum and plasma samples were stored at −80° C.

All enrolled subjects provided written informed consent before participation in the study and all aspects of study conduct were in accordance with Good Clinical Practice. Sera of Ebola virus survivors were collected in the context of the project EVIDENT (Ebola Virus Disease: Correlates of Protection, Determinants of Outcome, and Clinical Management) after obtaining informed consent and provided to use as control samples for anti-EBOV IgG titer and neutralizing activity.

### Expression and Purification of Ebolavirus GPs

Recombinant Ebolavirus GP constructs lacking the TM domain (Δ651-676, EBOV Makona (GenBank: KJ660347, EBOV Mayinga (GenBank: AF086833.2, BDBV (GenBank: FJ217161, SUDV Gulu (GenBank: AY729654.1) or TM and MLD domain [Δ313-464, EBOV Makona (GenBank: KJ660347)] as well as sGP (GenBank: KJ660347) were cloned into the pCAGGS backbone (Mittler, Schudt et al. 2018).

GP constructs were further modified to carry an Avi-tag as well as a His-tag for purification, and, except sGP, a GCN4 domain for GP complex formation at the C-terminal end (Corti, Misasi et al. 2016). HEK293F cells were maintained in FreeStyle Medium at 37° C. with 6% CO₂, shaking at 110 rpm. At a concentration of 0.8×10⁶ cells/ml transfection was performed using 25 kDa polyethylenimine (PEI) with 1 μg DNA/ml cell suspension.

After incubating for 7 days, supernatant was harvested, filtered (0.45 μm filter) and incubated with Protino Ni-NTA (nitrilotriacetic acid) agarose beads overnight. Beads were pelleted, transferred into a column and washed 1× with NPI-10 (H₂O, 50 mM NaH₂PO₄, 300 mM NaCl, 10 mM imidazole, pH=8) and 1× with NPI-20 (H2O, 50 mM NaH₂PO₄, 300 mM NaCl, 20 mM imidazole, pH=8) buffer. His-tagged glycoproteins were eluted by addition of NPI-100 (H2O, 50 mM NaH₂PO₄, 300 mM NaCl, 100 mM imidazole, pH=8) and buffer was exchanged to PBS using centrifugal filter units. All glycoproteins were sterile-filtered and stored at −80° C.

### Single Cell Sort of EBOV GPΔTM-Specific IgG⁺ B Cells

Peripheral blood mononuclear cells (PBMCs) were enriched for B lymphocytes by magnetic cell separation (MACS) with CD19-microbeads according to manufacturer's instruction. MACS LS separation columns (Miltenyi Biotec) were used to enrich labeled cells. B cells were spun and suspended in PBS with 2% FBS and 2 mM EDTA and subjected to fluorescence staining with DAPI, anti-huCD20-Alexa Fluor 700 (BD), anti-hulgG-APC, and EBOV GPΔTM-DyLight 488 (SEQ ID No. 158; Microscale Antibody Kit).

Cells were stained for 20 min at 4° C. and live CD20+ IgG+ EBOV GP+ cells were sorted using a FACSAriaIII according to the manufacturer's instructions in a single cell manner into 96-well plates. All wells contained 4 μl lysis buffer (0.5×PBS, 0.5 U/μl RNAsin, 0.5 U/μl RNaseOUT, and 10 mM DTT. After sorting, plates were immediately stored at −80° C. until further processing.

### Ig Heavy/Light Chain Amplification

Procedure for PCR amplification was performed as previously described (Tiller, Meffre et al. 2008, von Boehmer, Liu et al. 2016) but with various adjustments. Briefly, Random Hexamers, NP-40, and RNAseOUT were added to sorted cells in lysis buffer. The reaction was incubated for 1 min at 65° C. and cooled down for at least 2 min on ice. Afterwards, 1× Superscript IV RT buffer, 50 U/rxn SuperScript IV Reverse Transcriptase, dNTPs, DTT, RNAseOUT and RNasin were added and incubated 15 min at RT, 10 min at 50° C. and 10 min at 80° C.

cDNA was used to amplify light and heavy chains using PlatinumTaq HotStart polymerase according to manufacturer's protocol with 6% KB extender and 4 μl cDNA template. Amplification was achieved by sequential semi-nested PCRs using optimized and newly developed primer sets (Kreer, Döring et al., manuscript submitted). The results of the PCRs were analyzed by gel electrophoresis and products of correct sizes were subjected to Sanger sequencing.

### Sequence Analysis

Sequencing chromatograms were filtered for a mean Phred score of 28 and a minimal length of 240 nt. Remaining sequences were annotated with IgBLAST (Ye, Ma et al. 2013) and trimmed to extract only the variable region from FWR1 to the end of the J gene. Base calls within the variable region with a Phred score below 16 were masked and sequences with more than 15 masked nucleotides, stop codons, or frameshifts were excluded from further analyses.

In order to identify clonally related sequences within a single subject, we grouped all productive heavy chain sequences of that particular subject by identical V_{H} genes, determined the pairwise Levenshtein distance for their CDRH3s, and assigned an individual clone-number to sequence groups that share the same V_{H} gene and have a minimal CDRH3 identity of 75% (with respect to the shortest CDRH3).

100 rounds of input sequence randomization and clonal assignment were performed and the result with the lowest number of remaining unassigned (non-clonal) sequences was selected for downstream analyses. All clones were cross-validated by the investigators taking also into account shared mutations.

Cloning and Production of EBOV GP-Specific mAbs

Selected antibody sequences were cloned into mAb expression vectors by sequence- and ligation-independent cloning (SLIC) as previously described (von Boehmer, Liu et al. 2016). Briefly, amplicons for cloning were produced by PCR-amplification using Q5 High Fidelity polymerase (NEB) by standard PCR protocols and previously described primers (Tiller, Meffre et al. 2008). PCR products were purified (GeneJET Gel Extraction and DNA Cleanup Micro Kit, Macherey Nagel), cloned into expression vectors by SLIC reaction using T4 DNA polymerase (NEB) and transduced into E.coli DH5α. 4-8 colonies were evaluated by PCR and gel electrophoresis, and forwarded to Sanger sequencing. Plasmids with correct antibody sequences were amplified by midi preparation according to manufacturer's protocol and stored at 4° C. (short term) or −20° C.

Heavy and light chain-encoding plasmids were co-transfected into HEK293E cells with PEI reagent as described above. Cultures were harvested after 7 days, cell-free supernatants were filtered, and incubated with Protein G sepharose at 4° overnight. Beads were transferred to columns, washed 3× with PBS and antibodies were eluted with 0.1 M Glycine (pH=3). Subsequently, pH of eluate was neutralized with 1 M Tris-HCl (pH=8). Buffer was exchanged to PBS as described above.

### ELISA Analysis to Determine Antibody Binding Activity to GPs

ELISAs were conducted in high binding 96-well ELISA plates. Plates were coated with 2.5 μg/ml GPs at 4° C. overnight, washed 3× with PBST (PBS, 0.05% Tween-20) and blocked with PBST/2% BSA for 60 min at RT. mAbs were tested at 4- or 6-fold dilutions (1:3 or 1:5) with starting concentrations of 10 μg/ml. After incubation for 90 min at RT, plates were washed 3× and incubated with horseradish peroxidase-conjugated goat anti-human IgG antibody (1:2500 in PBST/2% BSA) for 60 min at RT.

Reaction was started with 2,2′-azino-bis(3-ethylResultsbenzothiazoline-6-sulphonic acid) solution (ABTS) and absorbance (OD 415 nm-695 nm) was measured with absorbance reader after fixed incubation times. For analysis, time points with overlapping standards curves were selected and ODs as well as EC₅₀ were determined. Antibodies were considered GP-reactive, if OD was above 0.2 at a 10 μg/ml mAb concentration and EC₅₀ did not exceed 15 μg/ml.

For assessment of EBOV GP-specificity of antibodies, EBOV GPΔTM was used to coat ELISA plates. To evaluate cross-reactivity, also GPs of Filoviruses BDBV, SUDV and MARV were tested.

### Neutralization Assays

Antibodies (starting at a concentration of 100 μg/ml) were serially diluted in 96-well culture plates in Dulbecco's Modified Eagle's Medium supplemented with 2% fetal calf serum, penicillin (50 units/ml), streptomycin (50 μg/ml) and L-glutamine (2 mM). 100 TCID₅₀ units of EBOV Mayinga (GenBank: NC_002549) and SUDV Boniface (GenBank: FJ968794.1) were added to the serum or antibody dilutions. Following incubation at 37° C. for 1 hour, Vero C1008 cells (ATCC® CRL-1586™; 10,000 cells per well) were added. Plates were then incubated at 37° C., 5% CO₂ and cytopathic effects (CPE) evaluated at day 7 post infection.

Neutralization was defined as clear reduction of CPE in serum or antibody dilutions compared to positive controls. Neutralization titers were calculated of four replicates as geometric mean titers (GMT; reciprocal value) or geometric mean concentrations in the case of monoclonal antibodies (GMC). The cut-off of the assay is determined by the first dilution of the respective serum or antibody. Neutralization assays were performed in the BSL-4 laboratory of the Institute of Virology, Philipps-University Marburg, Germany.

### REFERENCES

Bornholdt, Z. A., et al. (2016). “Isolation of potent neutralizing antibodies from a survivor of the 2014 Ebola virus outbreak.” Science 351(6277): 1078-1083.
Corti, D., et al. (2016). “Protective monotherapy against lethal Ebola virus infection by a potently neutralizing antibody.” Science 351(6279): 1339-1342.
Gaebler, C., et al. (2013). “Isolation of HIV-1-reactive antibodies using cell surface-expressed gp160Deltac (BaL.).” J Immunol Methods 397(1-2): 47-54.
Maruyama, T., et al. (1999). “Ebola Virus can be effectively neutralized by antibody produced in natural human infection.” J Virol.
Mittler, E., et al. (2018). “A Fluorescently Labeled Marburg Virus Glycoprotein as a New Tool to Study Viral Transport and Assembly.” J Infect Dis 218(suppl_5): S318-S326.
Tiller, T., et al. (2008). “Efficient generation of monoclonal antibodies from single human B cells by single cell RT-PCR and expression vector cloning.” Journal of Immunological Methods 329(1-2): 112-124.
von Boehmer, L., et al. (2016). “Sequencing and cloning of antigen-specific antibodies from mouse memory B cells.” Nat Protoc 11(10): 1908-1923.
Ye, J., et al. (2013). “IgBLAST: an immunoglobulin variable domain sequence analysis tool.” Nucleic Acids Res 41(Web Server issue): W34-40.

1. Method for obtaining human antibodies directed against an infectious pathogen, preferably against a virus, wherein the method comprises:
a) vaccination of a human subject with a vaccine comprising an antigenic protein derived from the infectious pathogen, wherein the vaccine comprises rVSV-ZEBOV,
b) isolating B lymphocytes exhibiting antibodies directed against the infectious pathogen on their cell surface and/or antibodies directed against the infectious pathogen from vaccinated subjects,
c) identifying B lymphocytes expressing antibodies directed against the infectious pathogen and/or antibodies directed against the infectious pathogen, on a single cell- or single antibody-basis, and
d) expressing monoclonal antibodies directed against the infectious pathogen.

2. Method according to claim 1, wherein the vaccination is an intentional vaccination of a human subject and/or wherein the infectious pathogen is a virus from the family Filoviridae, or from the genus Ebolavirus or Marburgvirus, or from the genus Ebolavirus, and/or wherein the antigenic protein derived from the infectious pathogen is an Ebolavirus glycoprotein.

3. Method according to claim 1, wherein the step of isolating B lymphocytes exhibiting antibodies directed against the infectious pathogen on their cell surface and/or antibodies directed against the infectious pathogen comprises first extracting B lymphocytes of the vaccinated subject, and then carrying out serial limiting dilutions, and/or wherein the step of isolating B lymphocytes exhibiting antibodies directed against the infectious pathogen on their cell surface and/or antibodies directed against the infectious pathogen comprises first extracting B lymphocytes of the vaccinated subject and then contacting the extracted B lymphocytes with a component of the infectious pathogen to allow selective sorting of the extracted B lymphocytes which exhibit antibodies directed against the infectious pathogen on their cell surface, wherein selective sorting comprises fluorescence-activated cell sorting (FACS), magnetic-activated cell sorting (MACS) or chromatographic separation, and wherein B lymphocytes exhibiting antibodies directed against the infectious pathogen on their cell surface and/or antibodies directed against the infectious pathogen are isolated from vaccinated subjects by
b.1) contacting extracted B lymphocytes with a component of the infectious pathogen labelled with a fluorescence marker, and
b.2) isolating B lymphocytes exhibiting antibodies directed against the infectious pathogen on their cell surface by fluorescence-activated cell sorting (FACS), in a single cell-manner.

4. Method according to claim 1, wherein the step of identifying B lymphocytes exhibiting antibodies directed against the infectious pathogen on their cell surface and/or antibodies directed against the infectious pathogen, comprises
c.1) amplifying nucleotide sequences coding for light and heavy chains of each individual antibody, and
c.2) sequencing the amplified nucleotide sequences of said light and heavy chains.

5. Method according to claim 1, wherein the step of expressing a monoclonal antibody directed against the infectious pathogen comprises:
d.1) cloning of the light and heavy chain sequences into a mAb expression vector,
d.2) transfecting the mAb expression vector into a suitable host cell,
d.3) culturing the suitable host cell, and
d.4) harvesting the expressed monoclonal antibody from the host cell supernatant.

6. Monoclonal human antibody or binding fragment thereof directed against an infectious pathogen, wherein the antibody is obtained by the method according to claim 1, wherein the infectious pathogen is a virus from the family Filoviridae, or from the genus Ebolavirus or Marburgvirus, or from the genus Ebolavirus.

7. Monoclonal human antibody or binding fragment thereof according to claim 6, wherein the antibody comprises:
I) an amino acid sequence encoded by the heavy chain V gene segment IGHV3-15 or an amino acid sequence being at least 80% identical thereto, and
II) an amino acid sequence encoded by the light chain V gene segment IGLV1-40 or an amino acid sequence being at least 80% identical thereto and/or wherein the antibody or binding fragment thereof exhibits reactivity with more than one virus from the family Filoviridae, or with at least two selected from the group comprising Bundibugyo virus, Marburg virus, Sudan virus, and Zaire Ebolavirus.

8. Monoclonal human antibody or binding fragment according to claim 6, wherein the antibody or binding fragment thereof exhibits neutralization of Ebolavirus in a neutralization assay at a concentration of 1 pg/mL or less, or at 0.74 pg/mL or less, or at 0.05 pg/mL or less, or at 0.01 pg/mL or less and/or wherein the antibody or binding fragment thereof exhibits superior neutralization of Ebolavirus in a neutralization assay in comparison to mAb100.

9. Monoclonal human antibody or binding fragment according to claim 6, wherein the antibody or binding fragment thereof comprises the heavy chain CDR1 to CDR3 and the light chain CDR1 to CDR3 amino acid sequence of one antibody from the group comprising 1T0129 (consisting of the heavy chain amino acid sequence of SEQ ID No. 1 and the light chain amino acid sequence of SEQ ID No. 2), 1T0139 (consisting of the heavy chain amino acid sequence of SEQ ID No. 3 and the light chain amino acid sequence of SEQ ID No. 4), 1T0201 (consisting of the heavy chain amino acid sequence of SEQ ID No. 5 and the light chain amino acid sequence of SEQ ID No. 6), 1T0221 (consisting of the heavy chain amino acid sequence of SEQ ID No. 7 and the light chain amino acid sequence of SEQ ID No. 8), 1T0225 (consisting of the heavy chain amino acid sequence of SEQ ID No. 9 and the light chain amino acid sequence of SEQ ID No. 10), 1T0227 (consisting of the heavy chain amino acid sequence of SEQ ID No. 11 and the light chain amino acid sequence of SEQ ID No. 12), 1T0248 (consisting of the heavy chain amino acid sequence of SEQ ID No. 13 and the light chain amino acid sequence of SEQ ID No. 14), 1T0321 (consisting of the heavy chain amino acid sequence of SEQ ID No. 15 and the light chain amino acid sequence of SEQ ID No. 16), 1T0325 (consisting of the heavy chain amino acid sequence of SEQ ID No. 17 and the light chain amino acid sequence of SEQ ID No. 18), 1T0371 (consisting of the heavy chain amino acid sequence of SEQ ID No. 19 and the light chain amino acid sequence of SEQ ID No. 20), 1T0451 (consisting of the heavy chain amino acid sequence of SEQ ID No. 21 and the light chain amino acid sequence of SEQ ID No. 22), 1T0455 (consisting of the heavy chain amino acid sequence of SEQ ID No. 23 and the light chain amino acid sequence of SEQ ID No. 24), 1T0465 (consisting of the heavy chain amino acid sequence of SEQ ID No. 25 and the light chain amino acid sequence of SEQ ID No. 26), 1T0473 (consisting of the heavy chain amino acid sequence of SEQ ID No. 27 and the light chain amino acid sequence of SEQ ID No. 28), 1T0552 (consisting of the heavy chain amino acid sequence of SEQ ID No. 29 and the light chain amino acid sequence of SEQ ID No. 30), 1T0655 (consisting of the heavy chain amino acid sequence of SEQ ID No. 31 and the light chain amino acid sequence of SEQ ID No. 32), 3T0123 (consisting of the heavy chain amino acid sequence of SEQ ID No. 33 and the light chain amino acid sequence of SEQ ID No. 34), 3T0202 (consisting of the heavy chain amino acid sequence of SEQ ID No. 35 and the light chain amino acid sequence of SEQ ID No. 36), 3T0215 (consisting of the heavy chain amino acid sequence of SEQ ID No. 37 and the light chain amino acid sequence of SEQ ID No. 38), 3T0245 (consisting of the heavy chain amino acid sequence of SEQ ID No. 39 and the light chain amino acid sequence of SEQ ID No. 40), 3T0253 (consisting of the heavy chain amino acid sequence of SEQ ID No. 41 and the light chain amino acid sequence of SEQ ID No, 42), 3T0258 (consisting of the heavy chain amino acid sequence of SEQ ID No. 43 and the light chain amino acid sequence of SEQ ID No. 44), 3T0265 (consisting of the heavy chain amino acid sequence of SEQ ID No. 45 and the light chain amino acid sequence of SEQ ID No. 46), 3T0331 (consisting of the heavy chain amino acid sequence of SEQ ID No. 47 and the light chain amino acid sequence of SEQ ID No. 48), 3T0338 (consisting of the heavy chain amino acid sequence of SEQ ID No. 49 and the light chain amino acid sequence of SEQ ID No. 50), 3T0350 (consisting of the heavy chain amino acid sequence of SEQ ID No. 51 and the light chain amino acid sequence of SEQ ID No. 52), 3T0405 (consisting of the heavy chain amino acid sequence of SEQ ID No. 53 and the light chain amino acid sequence of SEQ ID No. 54), 3T0415 (consisting of the heavy chain amino acid sequence of SEQ ID No. 55 and the light chain amino acid sequence of SEQ ID No. 56), 3T0468 (consisting of the heavy chain amino acid sequence of SEQ ID No. 57 and the light chain amino acid sequence of SEQ ID No. 58), 3T0478 (consisting of the heavy chain amino acid sequence of SEQ ID No. 59 and the light chain amino acid sequence of SEQ ID No. 60), 3T0553 (consisting of the heavy chain amino acid sequence of SEQ ID No. 61 and the light chain amino acid sequence of SEQ ID No. 62), 3T0611 (consisting of the heavy chain amino acid sequence of SEQ ID No. 63 and the light chain amino acid sequence of SEQ ID No. 64), 3T0650 (consisting of the heavy chain amino acid sequence of SEQ ID No. 65 and the light chain amino acid sequence of SEQ ID No. 66), 3T0673 (consisting of the heavy chain amino acid sequence of SEQ ID No. 67 and the light chain amino acid sequence of SEQ ID No. 68), 4m0333 (consisting of the heavy chain amino acid sequence of SEQ ID No. 69 and the light chain amino acid sequence of SEQ ID No. 70), 4m0368 (consisting of the heavy chain amino acid sequence of SEQ ID No. 71 and the light chain amino acid sequence of SEQ ID No. 72), 4m0373 (consisting of the heavy chain amino acid sequence of SEQ ID No. 73 and the light chain amino acid sequence of SEQ ID No. 74), 4T0115 (consisting of the heavy chain amino acid sequence of SEQ ID No. 75 and the light chain amino acid sequence of SEQ ID No. 76), 4T0154 (consisting of the heavy chain amino acid sequence of SEQ ID No. 77 and the light chain amino acid sequence of SEQ ID No. 78), 4T0159 (consisting of the heavy chain amino acid sequence of SEQ ID No. 79 and the light chain amino acid sequence of SEQ ID No. 80), 4T0176 (consisting of the heavy chain amino acid sequence of SEQ ID No. 81 and the light chain amino acid sequence of SEQ ID No. 82), 4T0182 (consisting of the heavy chain amino acid sequence of SEQ ID No. 83 and the light chain amino acid sequence of SEQ ID No. 84), 4T0243 (consisting of the heavy chain amino acid sequence of SEQ ID No. 85 and the light chain amino acid sequence of SEQ ID No. 86), 4T0262 (consisting of the heavy chain amino acid sequence of SEQ ID No. 87 and the light chain amino acid sequence of SEQ ID No. 88), 4T0284 (consisting of the heavy chain amino acid sequence of SEQ ID No. 89 and the light chain amino acid sequence of SEQ ID No. 90), 4T0306 (consisting of the heavy chain amino acid sequence of SEQ ID No. 91 and the light chain amino acid sequence of SEQ ID No. 92), 4T0344 (consisting of the heavy chain amino acid sequence of SEQ ID No. 93 and the light chain amino acid sequence of SEQ ID No. 94), 4T0365 (consisting of the heavy chain amino acid sequence of SEQ ID No. 95 and the light chain amino acid sequence of SEQ ID No. 96), 4T0412 (consisting of the heavy chain amino acid sequence of SEQ ID No. 97 and the light chain amino acid sequence of SEQ ID No. 98), 4T0423 (consisting of the heavy chain amino acid sequence of SEQ ID No. 99 and the light chain amino acid sequence of SEQ ID No. 100), 4T0427 (consisting of the heavy chain amino acid sequence of SEQ ID No. 101 and the light chain amino acid sequence of SEQ ID No. 102), 4T0429 (consisting of the heavy chain amino acid sequence of SEQ ID No. 103 and the light chain amino acid sequence of SEQ ID No. 104), 4T0434 (consisting of the heavy chain amino acid sequence of SEQ ID No. 105 and the light chain amino acid sequence of SEQ ID No. 106), 4T0444 (consisting of the heavy chain amino acid sequence of SEQ ID No. 107 and the light chain amino acid sequence of SEQ ID No. 108), 4T0452 (consisting of the heavy chain amino acid sequence of SEQ ID No. 109 and the light chain amino acid sequence of SEQ ID No. 110), 4T0525 (consisting of the heavy chain amino acid sequence of SEQ ID No. 111 and the light chain amino acid sequence of SEQ ID No. 112), 4T0541 (consisting of the heavy chain amino acid sequence of SEQ ID No. 113and the light chain amino acid sequence of SEQ ID No. 114), 4T0570 (consisting of the heavy chain amino acid sequence of SEQ ID No. 115 and the light chain amino acid sequence of SEQ ID No. 116), 4T0578 (consisting of the heavy chain amino acid sequence of SEQ ID No. 117 and the light chain amino acid sequence of SEQ ID No. 118), 4T0657 (consisting of the heavy chain amino acid sequence of SEQ ID No. 119 and the light chain amino acid sequence of SEQ ID No. 120), 4T0726 (consisting of the heavy chain amino acid sequence of SEQ ID No. 121 and the light chain amino acid sequence of SEQ ID No. 122), 4T0764 (consisting of the heavy chain amino acid sequence of SEQ ID No. 123 and the light chain amino acid sequence of SEQ ID No. 124), 4T0784 (consisting of the heavy chain amino acid sequence of SEQ ID No. 125 and the light chain amino acid sequence of SEQ ID No. 126), 5T0107 (consisting of the heavy chain amino acid sequence of SEQ ID No. 127 and the light chain amino acid sequence of SEQ ID No. 128), 5T0180 (consisting of the heavy chain amino acid sequence of SEQ ID No. 129 and the light chain amino acid sequence of SEQ ID No. 130), 5T0202 (consisting of the heavy chain amino acid sequence of SEQ ID No. 131 and the light chain amino acid sequence of SEQ ID No. 132), 5T0209 (consisting of the heavy chain amino acid sequence of SEQ ID No. 133 and the light chain amino acid sequence of SEQ ID No. 134), 5T0223 (consisting of the heavy chain amino acid sequence of SEQ ID No.,135 and the light chain amino acid sequence of SEQ ID No. 136), 5T0257 (consisting of the heavy chain amino acid sequence of SEQ ID No. 137 and the light chain amino acid sequence of SEQ ID No. 138), 5T0278 (consisting of the heavy chain amino acid sequence of SEQ ID No. 139 and the light chain amino acid sequence of SEQ ID No. 140), 5T0337 (consisting of the heavy chain amino acid sequence of SEQ ID No. 141 and the light chain amino acid sequence of SEQ ID No. 142), 5T0376 (consisting of the heavy chain amino acid sequence of SEQ ID No. 143 and the light chain amino acid sequence of SEQ ID No. 144), 5T0378 (consisting of the heavy chain amino acid sequence of SEQ ID No. 145 and the light chain amino acid sequence of SEQ ID No. 146), 5T0404 (consisting of the heavy chain amino acid sequence of SEQ ID No. 147 and the light chain amino acid sequence of SEQ ID No. 148), 5T0406 (consisting of the heavy chain amino acid sequence of SEQ ID No. 149 and the light chain amino acid sequence of SEQ ID No. 150), 5T0420 (consisting of the heavy chain amino acid sequence of SEQ ID No. 151 and the light chain amino acid sequence of SEQ ID No. 152), 5T0451 (consisting of the heavy chain amino acid sequence of SEQ ID No. 153 and the light chain amino acid sequence of SEQ ID No. 154), 5T0465 (consisting of the heavy chain amino acid sequence of SEQ ID No. 155 and the light chain amino acid sequence of SEQ ID No. 156), or of one antibody from the group comprising 1T0325, 1T0451, 1T0451, 1T0655, 3T0123, 3T0253, 3T0553, 4T0243, 4T0306, 4T0570, 4T0726, 4T0764, 4T0784, 5T0180, 5T0223, 5T0278, 5T0337, 5T0451, or wherein the antibody or binding fragment thereof comprises the heavy chain CDR1 to CDR3 and the light chain CDR1 to CDR3 amino acid sequence of one of the antibodies selected from 3T0331 (consisting of the heavy chain amino acid sequence of SEQ ID No. 47 and the light chain amino acid sequence of SEQ ID No. 48) or 4T0243 (consisting of the heavy chain amino acid sequence of SEQ ID No. 85 and the light chain amino acid sequence of SEQ ID No. 86), or of antibody 3T0331.

10. Monoclonal human antibody or binding fragment according to claim 6, wherein the antibody or binding fragment thereof comprises the combination of the heavy chain and the light chain of one antibody selected from the group comprising 1T0129 (SEQ ID No. 1 and 2), 1T0139 (SEQ ID No. 3 and 4), 1T0201 (SEQ ID No. 5 and 6), 1T0221 (SEQ ID No. 7 and 8), 1T0225 (SEQ ID No. 9 and 10), 1T0227 (SEQ ID No. 11 and 12), 1T0248 (SEQ ID No. 13 and 14), 1T0321 (SEQ ID No. 15 and 16), 1T0325 (SEQ ID No. 17 and 18), 1T0371 (SEQ ID No. 19 and 20), 1T0451 (SEQ ID No. 21 and 22), 1T0455 (SEQ ID No. 23 and 24), 1T0465 (SEQ ID No. 25 and 26), 1T0473 (SEQ ID No. 27 and 28), 1T0552 (SEQ ID No. 29 and 30), 1T0655 (SEQ ID No. 31 and 32), 3T0123 (SEQ ID No. 33 and 34), 3T0202 (SEQ ID No. 35 and 36), 3T0215 (SEQ ID No. 37 and 38), 3T0245 (SEQ ID No. 39 and 40), 3T0253 (SEQ ID No. 41 and 42), 3T0258 (SEQ ID No. 43 and 44), 3T0265 (SEQ ID No. 45 and 46), 3T0331 (SEQ ID No. 47 and 48), 3T0338 (SEQ ID No. 49 and 50), 3T0350 (SEQ ID No. 51 and 52), 3T0405 (SEQ ID No. 53 and 54), 3T0415 (SEQ ID No. 55 and 56), 3T0468 (SEQ ID No. 57 and 58), 3T0478 (SEQ ID No. 59 and 60), 3T0553 (SEQ ID No. 61 and 62), 3T0611 (SEQ ID No. 63 and 64), 3T0650 (SEQ ID No. 65 and 66), 3T0673 (SEQ ID No. 67 and 68), 4m333 (SEQ ID No. 69 and 70), 4m0368 (SEQ ID No. 71 and 72), 4m0373 (SEQ ID No. 73 and 74), 4T0115 (SEQ ID No. 75 and 76), 4T0154 (SEQ ID No. 77 and 78), 4T0159 (SEQ ID No. 79 and 80), 4T0176 (SEQ ID No. 81 and 82), 4T0182 (SEQ ID No. 83 and 84), 4T0243 (SEQ ID No. 85 and 86), 4T0262 (SEQ ID No. 87 and 88), 4T0284 (SEQ ID No. 89 and 90), 4T0306 (SEQ :ID No. 91 and 92), 4T0344 (SEQ ID No. 93 and 94)., 4T0365 (SEQ ID No. 95 and 96), 4T0412 (SEQ ID No. 97 and 98), 4T0423 (SEQ ID No. 99 and 100), 4T0427 (SEQ ID No. 101 and 102), 4T0429 (SEQ ID No. 103 and 104), 4T0434 (SEQ ID No. 105 and 106), 4T0444 (SEQ ID No. 107 and 108), 4T0452 (SEQ ID No. 109 and 110), 4T0525 (SEQ ID No. 111 and 112), 4T0541 (SEQ ID No. 113 and 114), 4T0570 (SEQ ID No. 115 and 116), 4T0578 (SEQ ID No. 117 and 118), 4T0657 (SEQ ID No. 119 and 120), 4T0726 (SEQ ID No. 121 and 122), 4T0764 (SEQ ID No. 123 and 124), 4T0784 (SEQ ID No. 125 and 126), 5T0107 (SEQ ID No. 127 and 128), 5T0180 (SEQ ID No. 129 and 130), 5T0202 (SEQ ID No. 131 and 132), 5T0209 (SEQ ID No. 133 and 134), 5T0223 (SEQ ID No. 135 and 136). 5T0257 (SEQ ID No. 137 and 138), 5T0278 (SEQ ID No. 139 and 140), 5T0337 (SEQ ID No. 141 and 142), 5T0376 (SEQ ID No. 143 and 144), 5T0378 (SEQ ID No. 145 and 146), 5T0404 (SEQ ID No. 147 and 148), 5T0406 (SEQ ID No. 149 and 150), 5T0420 (SEQ ID No. 151 and 152), 5T0451 (SEQ ID No. 153 and 154), 5T0465 (SEQ ID No. 155 and 156), or of one antibody from the group comprising 1T0325, 1T0451, 1T0473, 1T0655, 3T0123, 3T0253, 3T0553, 4T0243, 4T0306, 4T0570, 4T0726, 4T0764, 4T0784, 5T0180, 5T0223, 5T0278, 5T0337, 5T0451, or wherein the antibody or binding fragment thereof comprises the combination of the heavy chain and the light chain of one of the antibodies selected from 3T0331 (SEQ ID No. 47 and 48) or 4T0243 (SEQ ID No. 85 and 86), or of 3T0331.

11. Pharmaceutical composition comprising a monoclonal human antibody or binding fragment thereof according to claim 6 and at least one pharmaceutically acceptable excipient, wherein the pharmaceutical composition is a vaccination composition for a human subject.

12. Kit comprising a monoclonal human antibody or binding fragment thereof according to claim 6 and a container.

13. Monoclonal human antibody or binding fragment thereof according to claim 6, pharmaceutical composition or kit for use as a medicament, or for use as a vaccine.

14. Monoclonal human antibody or binding fragment thereof according to claim 6, pharmaceutical composition, or kit for use in the treatment or prevention of a disease caused by the infectious pathogen in human subjects, or for use in the treatment or prevention of Ebolavirus-caused disease in human subjects.


SEQUENCE LISTING (158 sequences)

SEQ ID NO: 1 - protein, 117 aa, Homo sapiens
  Heavy chain of antibody 1T0129
QVQLVQSGAEAKKPGASVRVSCQTSGYTFTGHYLHWVRQAPGQGLEWLGWINPSTGGTFYAPKFKDRVDMTRDTSTTTAYMNLSSLTSDDTAVYYCGTGIPSDIDYWGQGTLVTVSS

SEQ ID NO: 2 - protein, 108 aa, Homo sapiens
  Light chain of antibody 1T0129
DIVMTQFPDFVAVSPGERATISCESSQSVLYQSVAWYQHKPGQPPKLIISWASRRGSGVPNRFSGSGSGTNFSLTISSLRPEDVAVYYCQQYYNTPRTFGQGTKLEIK

SEQ ID NO: 3 - protein, 123 aa, Homo sapiens
  Heavy chain of antibody 1T0139
QVQLQESGPGLVKPSETLSLTCIVSGGSLSSYYWSWVRQPAGKGLQWIGHIYTTGSTNYNPSLKSRVTMSIDTSKNQFSLRLSSVTAADTAVYYCARESYRYNAYDIHPFDYWGQGTLVTVSS

SEQ ID NO: 4 - protein, 106 aa, Homo sapiens
  Light chain of antibody 1T0139
SYELTQSSSVSVSPGQTASITCSGDKLGDNYVCWYQQKPGQSPTLVIYQDNRRPSGIPERFSGSNSGNTATLTIRGTQALDEADYYCQAWASNTVVFGGGTKLTVL

SEQ ID NO: 5 - protein, 124 aa, Homo sapiens
  Heavy chain of antibody 1T0201
EVQLVESGGGLVKPGGSLRLSCAAAGFSFSKAWMNWVRQAPGKGLEWVGRIKSKTDGGTTDYTAPVKGRFIISRDDSKNMLYLQMNSLKAEDTAVYWCTTRVDMEWGKYGTDVWGQGTTVTVSS

SEQ ID NO: 6 - protein, 112 aa, Homo sapiens
  Light chain of antibody 1T0201
QSVLTQPPSVSGAPGQRVTISCTGSSSNIGAGYDVHWYQKLPGTAPKLLIYDKNIRPSGVPDRFSGSKSGVSASLAITGLQAEDEADYYCQSYDSRLSDSWVFGGGTKLTVL

SEQ ID NO: 7 - protein, 121 aa, Homo sapiens
  Heavy chain of antibody 1T0221
QVQLVQSGAEVKKPGASVKVSCKASGYTFTHYGISWVRQAPGQGLEWMGWISGYNRNTKFAQNFQGRVTLTTDTSTSTAYLELRSLRSDDTAVYYCARHVTMILDGWFDPWGQGTLVTVSS

SEQ ID NO: 8 - protein, 108 aa, Homo sapiens
  Light chain of antibody 1T0221
EIVLTQSPGTLSLSPGERATLSCRASQNVSSSYLAWYQQKPGQAPRLLIYGASNRATGIPDRFSGSGSGTDFTLTISRLEPEDFAVYSCQQYGSSPLTFGGGTKVEIK

SEQ ID NO: 9 - protein, 118 aa, Homo sapiens
  Heavy chain of antibody 1T0225
QVQLQESGSGLVKPSQTLSLTCAVSGGAITSGGFSWSWIRQPPGKGLEWIGYIYPSGSAYYNPSLKSRVTMSVDTSKKQFSLKLNSVTAADTAVYYCARDPGGPFDHWGQGTLVTVSS

SEQ ID NO: 10 - protein, 109 aa, Homo sapiens
  Light chain of antibody 1T0225
QSALTQPASVSGSPGQSITISCTATSSDLDDYNYVSWYQQHPGKGPRLIFYDVNNRPSGVSNRFSGSKSGDTASLTISGLQPEDEADYYCSSYTSNFSVVFGGGTKLTV

SEQ ID NO: 11 - protein, 124 aa, Homo sapiens
  Heavy chain of antibody 1T0227
EVQLVESGGGLVKPGGSLRLSCAASGFTFRNAWMNWVRQAPGKGLEWVGRIKSRADGGPTDYAAPVKGRFTISRDDSKNTLYLQMNSLKSEDTAVYYCTTHVPDYNSAYYWVYWGQGTLVTVSS

SEQ ID NO: 12 - protein, 112 aa, Homo sapiens
  Light chain of antibody 1T0227
QSVLTQPPSVSGAPGQRVTISCTGSSSNIGAGYDVYWYQQLPGTAPKLIIYGNNNRPSGVPDRFSASKSGPSASLAITGLQAEDEADYYCQSYDIRLSDNYVFGTGTKVTVL

SEQ ID NO: 13 - protein, 123 aa, Homo sapiens
  Heavy chain of antibody 1T0248
EVQLLESGGGLVQPGGSLRLSCAASGFMFNNDAMSWVRQAPGKGLEWVSGISGGGTITHYADSVKGRFTISRDNSNNTLYLQMNSLRAEDTAIYYCAKDKGPRATLGAILDSWGQGTLVTVSS

SEQ ID NO: 14 - protein, 113 aa, Homo sapiens
  Light chain of antibody 1T0248
DIVMTQSPDSLAVSLGERATINCKSSQSVLYNSDNKNYLAWYQQRPGLPPKLLIYWASTRESGVPDRFSGSGSGTDFTLTISSLQAEDVAVYYCQQYFGTPLTFGPGTKVDIK

SEQ ID NO: 15 - protein, 123 aa, Homo sapiens
  Heavy chain of antibody 1T0321
QVQLVQSGADVKKPGASVKVSCRASGYKFTDFYIHWVRQAPGQGLEWMGWINPNSGATNFPQKFQGRVAMTRDTSISTAYLELSRLRSDDTAVYYCATPTMTTRSYYYHMDVWGQGTTVTVSS

SEQ ID NO: 16 - protein, 112 aa, Homo sapiens
  Light chain of antibody 1T0321
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMHALQTPWTFGQGTKVEIK

SEQ ID NO: 17 - protein, 123 aa, Homo sapiens
  Heavy chain of antibody 1T0325
EVQLLESGGGLVQPGGSLRLSCAASGFPFRNYAMNWVRQAPGKGLEWVASISGSGNRTHYADALKGRFTISRDNFKNTLFLQMNSLRADDTAVYYCAKEREVAFVPGAVFNYWGQGTLVTVSS

SEQ ID NO: 18 - protein, 113 aa, Homo sapiens
  Light chain of antibody 1T0325
DIVMTQSPDSLAVSLGERATINCKSSQRILYSSNNKNYLTWYQQKPGQPPKLLIYWASTRESGVPDRFSGSGSGTDFTLTISSLQAEDVAVYYCQQYYSLPLTFGGGTKVEIK

SEQ ID NO: 19 - protein, 117 aa, Homo sapiens
  Heavy chain of antibody 1T0371
QVQLVQSGAEVKKPGSSVKVSCKASGGTFSRFSISWVRQAPGQGLEWMGRIIPTLGIANYAQKFQGRATLTADKSTSTAFMELTSLRSEDTAVYYCATLEMATLFAWGQGTLVTVSS

SEQ ID NO: 20 - protein, 107 aa, Homo sapiens
  Light chain of antibody 1T0371
DIQMTQSPSSLSASVGDRVTITCRASQGITNYLAWFQQKPGKAPKSLIYAASSLQSGVPSKFSGSGSGTDFTLTISSLQPEDFATYYCQQYNSYPFTFGGGTKVEIK

SEQ ID NO: 21 - protein, 121 aa, Homo sapiens
  Heavy chain of antibody 1T0451
QVQLVQSGAEVKKPGASVKISCEASGYNFNGYVIDWVRQAPGQRLEWMGWINPGNGNTKFSPKFQGRVTFTRAASASTAYMELSSLTSEDTALYFCARGRGTSYYYAFDVWGQGTTVTVSS

SEQ ID NO: 22 - protein, 108 aa, Homo sapiens
  Light chain of antibody 1T0451
DIQMTQFPSSLSASVGDRVTISCRASQNISNYLNWYQQKPGKAPKFLIYAASNLQSGVPSRFSGSGSGTGFTLTISNLQPEDFAIYYCQQTYRSPLYTFGQGTKLEIK

SEQ ID NO: 23 - protein, 114 aa, Homo sapiens
  Heavy chain of antibody 1T0455
EVQLVESGGGLVKPGESLRLSCAVSGLTFSNAWMNWVRQAPGKGLEWVGRIKSKSDGGAADYAAPVNARFTISRDDSKKTLYLQMNSLKTEDTGFYYCTTLYHSGQGTLVTVSS

SEQ ID NO: 24 - protein, 112 aa, Homo sapiens
  Light chain of antibody 1T0455
QSVLTQPPSVSGAPGQRVTISCTGSSSNIGAGYDVHWYQQLPGTAPKFVIYGNNNRPSGVPDRFSGSKSGTSASLAITGLQVEDEGDYYCQSYDSRLSDHVVFGGGTKLTVL

SEQ ID NO: 25 - protein, 121 aa, Homo sapiens
  Heavy chain of antibody 1T0465
QVQLVQSGAEVKKPGASVKVSCKASGYTFTRFGINWVRQAPGQGLEWMGWISVYNGKTDYAQNVQGRVTMTTDTATSTAYMELRSLRSDDTAVYYCARDITMVRGGWLDYWGQGTLITVSS

SEQ ID NO: 26 - protein, 106 aa, Homo sapiens
  Light chain of antibody 1T0465
EIVMTQSPATLSVSPGERATLSCRASQSIDTNLAWYQQKPGQAPRLLIYYASTRATGTPARFSGSGSGTEFALTISSLQSEDFAVYYCQQYNTWATFGQGTKLEIK

SEQ ID NO: 27 - protein, 126 aa, Homo sapiens
  Heavy chain of antibody 1T0473
QVQLVQSGAEVKKPGASVKVSCKASGYTFPSYEINWVRQATGQGLEWMGWMDPKSGKTGYAQKFQGRVTMTRNPSISTAYLELSSLRSEDSAVYYCARGNQWFGELLSSYYGLDVWGQGTTVTVSS

SEQ ID NO: 28 - protein, 108 aa, Homo sapiens
  Light chain of antibody 1T0473
EIVLTQSPGTLSLSPGEGATLSCRASQSVSSSFLTWYHQKPGQAPRLLIYGASNRATGVPDRFSGGGSGTDFTLTISRLEPEDFGVYYCQQYGSSLITFGPGTKVDIK

SEQ ID NO: 29 - protein, 120 aa, Homo sapiens
  Heavy chain of antibody 1T0552
EVQLLESGGGLIQPGGSLRLSCAASGFTFSNFPMSWVRQAPGKGLEWVAAISGSGASIYYAESVRGRFTIFRDNSKNTLYLQMNSLRADDTAVYYCAKFSQLFGYAFDIWGQGTMVTVSS

SEQ ID NO: 30 - protein, 108 aa, Homo sapiens
  Light chain of antibody 1T0552
EIVLTQSPGTLSLSPGERATLSCRASQSVTRSWFAWYQQKPGQAPRLLIYGASNRAPGIPDRFSGSGSGADFTLTISGLEPEDFAVYYCQQYGNSPWTFGQGTKVEIK

SEQ ID NO: 31 - protein, 128 aa, Homo sapiens
  Heavy chain of antibody 1T0655
QVQLVQSGAEVKKPGASVKVSCKASGYTFTSYYMHWVRQVPGQGLEWMGIIKAGNGGTNYAQNFQGRITVTRDTSTSTVYMELSSLRSEDTAVYYCARDRQWLPLNGYSYFNYGMDVWGQGTTVTVSS

SEQ ID NO: 32 - protein, 107 aa, Homo sapiens
  Light chain of antibody 1T0655
DIQMTQSPSSLSASVGDKITITCRASQSISTYLNWYQQRPGRAPKLLIFGASSLQSGVPSRFSGSGSGTVFTLSISSLQPDDFGSFYCQQSFSSPRTFGQGTKVEIK

SEQ ID NO: 33 - protein, 126 aa, Homo sapiens
  Heavy chain of antibody 3T0123
QVQLVQSGSEVKKPGASVKVSCQASGYRFANYGISWVRQAPGQGLEWMGWISPLKRDPDYAQNFQGRVTMSRDTSTNTAYMELRGLTSGDTAVYFCARGLGDSYSNAWFVYVFDYWGQGSPVTVSS

SEQ ID NO: 34 - protein, 106 aa, Homo sapiens
  Light chain of antibody 3T0123
EIVMTQSPATLSVSPGERVTLSCRTSQRIHTNLAWYQQKSGQAPRLVMYDAFTRAPGVPARFSGGGSETEFTLTISSLQSEDFALYYCQQYSQRPTFGLGTKLEIK

SEQ ID NO: 35 - protein, 118 aa, Homo sapiens
  Heavy chain of antibody 3T0202
EVQLVESGGGLVQPGGSLRLSCAASGFKFRSYDMHWVRQRSGEGLEWVAAIGTAADTYYLGSVRGRFTISRENAKNSLYLQMDSLGAGDTAVYYCARAAFGAVFFDYWGQGTLVTVSS

SEQ ID NO: 36 - protein, 108 aa, Homo sapiens
  Light chain of antibody 3T0202
EIVLTQSPGTLSLSSGERATLSCRASQSISSSYLVWYQQKLGQAPRLLIYGASRRATGIPDRFSGSGSGTDFTLTISRLEPEDFAVYYCQQYGNSPITFGQGTRLEIK

SEQ ID NO: 37 - protein, 122 aa, Homo sapiens
  Heavy chain of antibody 3T0215
EVQLVESGGGSIQPGGSLRLSCTASGFPFSAHSFNWVRQAPGKGLEWISYINSSRTSIKYADSVKGRFTVSRDNAKQSLYLQMNSLGVEDTALYYCARDTLEAGSWYPFDKWGHGTLVTVSS

SEQ ID NO: 38 - protein, 108 aa, Homo sapiens
  Light chain of antibody 3T0215
SYELTQPPSVSVSPGQTARITCSGDALPQKYVYWYQQKSVQAPVLVIYEDSKRPSGIPERFSGSSSGTLATLRISGAQVEDEADYYCYSTDSSGTQRVFGGGTKLTVL

SEQ ID NO: 39 - protein, 120 aa, Homo sapiens
  Heavy chain of antibody 3T0245
EVQLVESGGDLVKPGGSLRLSCEASGFTFTNAWMNWVRQSPGKGLEWIGRVKSKSDGGAADYAAPVKGRFTISRDDSKNSVFLQMRSLKPEDTGVYYCATRVHYNGMDVWGRGTTVTVSS

SEQ ID NO: 40 - protein, 111 aa, Homo sapiens
  Light chain of antibody 3T0245
QSVLTQPPSVSGAPGQRVTISCAGSGSNIGAGYDVHWYQQLPGTAPKLLIYANHNRPSGVPDRFSGSKSGSSASLAITGLQAEDEADYYCQSYDSSLRDACVFGGGTKLTV

SEQ ID NO: 41 - protein, 119 aa, Homo sapiens
  Heavy chain of antibody 3T0253
EVQLVESGGGLVKPGRSLRISCAASGITFNKAWMNWVRLAPGKGLEWVGRIKSNGDGGKRDYAAPVTGRFTISRDDSKNTLYLQMNSLKTEDTAVYFRTTRHHRGMDVWGQGTTVTVSS

SEQ ID NO: 42 - protein, 112 aa, Homo sapiens
  Light chain of antibody 3T0253
QSVLTQPPSVSGAPGQRVTISCTGSSSNIGAGYDVHWYQHLPGAAPKLLIYGDGSRPSGVPDRFSASKSGTSASLAITGLQAEDEADYYCQSYDSSLSDAVVFGGGTKLTVL

SEQ ID NO: 43 - protein, 119 aa, Homo sapiens
  Heavy chain of antibody 3T0258
QVQLVQSGAEVKQPGASVKVTCKASGSTRSSYDLIWVRQAPGQGLELMGRVNTHNGNTDYAQKFQGRVTMTTDTSTTTAHMELRNLRSDDTAVYYCAWRRSSWFAFDVWGQGTAVTVSS

SEQ ID NO: 44 - protein, 108 aa, Homo sapiens
  Light chain of antibody 3T0258
EIVLTQSPGTLSLSPGERGTLSCRASQSISGNYLGWYQQSPGQAPRLLIYGASNRATGVPDRFSGSGSGTDFTLTISRLEPEDFAVYYCQQYVSSPYTFGQGTKLEIK

SEQ ID NO: 45 - protein, 117 aa, Homo sapiens
  Heavy chain of antibody 3T0265
EVQLVESGGNLAKPGGSLRLSCAASGFTFSDAWMTWVRQAPGKGLEWVGRIKSNADGGATEYAAPVRGRFTISRDDSRSSLFLQMNSLKTEDTAVYYCLTPPPRRYWGQGILVTVSS

SEQ ID NO: 46 - protein, 111 aa, Homo sapiens
  Light chain of antibody 3T0265
QSVLTQPPSVSGAPGQRVAISCTGSYSNIGAGYDVHWYQQFPGAAPKLLIYAYTNRPAGVPDRFSGSKSGSSASLVISGLQADDEADYYCQSYDARLRDGEVFGGGTKLTV

SEQ ID NO: 47 - protein, 121 aa, Homo sapiens
  Heavy chain of antibody 3T0331
EVQLLESGGGLVQPGGSLRLSCEASGFPLRDYAMSWVRQAPGRGLQWVSTIGGNDNAANYADSVKGRFTVSRDNSKSTIYLQMNSLRAEDTALYFCAKSVRLSRPSPFDLWGQGSLVTVSS

SEQ ID NO: 48 - protein, 107 aa, Homo sapiens
  Light chain of antibody 3T0331
EIVLTQSPATLSLSPGERATLSCRASQSVSTYLAWYQHQPGQAPRLLIYEASNRATGIPARFSGSGSGTEFTLTISSLEPEDVAVYYCQQRASWPLTFGGGTKVEIK

SEQ ID NO: 49 - protein, 125 aa, Homo sapiens
  Heavy chain of antibody 3T0338
EVQLVESGGALVKPGGSLRLSCAASGFSFKTAWMNWVRQAPGKGLEWVGRIKSRADGGTSDYSASVKGRFTFSRDDSKNTLYLQMTSLKTEDTGMYYCTTLNLGGGFPGEWRSVWGQGTTVTVSS

SEQ ID NO: 50 - protein, 112 aa, Homo sapiens
  Light chain of antibody 3T0338
QSVLTQPPSVSGAPGQRVTISCTGSYSNIGSGYDVLWYQQLPGAAPKLLIYGNNNRPSGVPDRFSGSKSGTSASLAITELQAEDEGDYYCQSYDSRLRDSWVFGGGTRLTVL

SEQ ID NO: 51 - protein, 122 aa, Homo sapiens
  Heavy chain of antibody 3T0350
EVQLLESGGGLVQPGGSLRLSCTASGFAFGNYAMNWVRQAPGKGLAWVAGVTGSGGTTIYADSVKGRFTISKDISKNTVYLQMSSLRAEDTAVYYCAKDLKGNWNDGPFDYWGQGALVTVSS

SEQ ID NO: 52 - protein, 112 aa, Homo sapiens
  Light chain of antibody 3T0350
QSVLTQPPSVSAAPGQKVTISCSGSRSNIGTNYVSWYQQLPGTAPKLLIYDNDKRPSGIPDRFSGSKSGTSATLGITGLQTGDEAEYYCGTWGNSLSGPEWVFGGGTRVTVL

SEQ ID NO: 53 - protein, 117 aa, Homo sapiens
  Heavy chain of antibody 3T0405
EVQLVESGGGVVQPGRSLRLSCAASGFTFSDYGMHWVRQAPGKGLEWAAVILYDGSKEYYADSVKGRFTISRDNSKNTLYLQMNSLRAEDTAVYYCAANYRDSELEWGQGTLVTVSS

SEQ ID NO: 54 - protein, 112 aa, Homo sapiens
  Light chain of antibody 3T0405
DVVMTQSPLSLTVTLGQPASISCRSSQSLLHSDGNTYLNWFHQRPGQSPRRLIFKVSDRDSGVPERFSGRGSDTDFTLKISRVEAEDVGVYYCMQATLWPWTFGQGTKVHVK

SEQ ID NO: 55 - protein, 113 aa, Homo sapiens
  Heavy chain of antibody 3T0415
EVQLLESGGGLVQPGGSLRLSCAASGFAFSTYAMSWVRQAPGKGLEWVSAVGGGGTNTHYADSVKGRFTISRDNSQNTLYLQMDSLRVDDTAVYYCAINGGVWGQGTAVTVSS

SEQ ID NO: 56 - protein, 113 aa, Homo sapiens
  Light chain of antibody 3T0415
DIVMTQSPDSLTVSLGERATITCKSSRSVFYSSNNKNYLAWYQQKPGQPPKLLISWASTRESGVPDRFSGSGSGTEFSLTISSLQAEDVAVYYCQQHYATRVTFGGGTKVEIK

SEQ ID NO: 57 - protein, 118 aa, Homo sapiens
  Heavy chain of antibody 3T0468
EVQLVESGGHLIQPGGSLRLSCAASGFTFRIYDMHWVRQGRRKGLEWVSAIGAAGDTYYSDSVKGRFTISRENAKSSLYLQMNSLSVGDTAVYYCARAAFGAVYYDNWGQGILVTVSS

SEQ ID NO: 58 - protein, 108 aa, Homo sapiens
  Light chain of antibody 3T0468
EIVLTQSPGTLSLSPGERATLSCRASENVDSRFLVWYQQKVGQAPRLLIYGASRRATGIPDRFSGSGSGTDFALTISRLEPEDLAVYYCQQYGRSPITFGQGTRLEIK

SEQ ID NO: 59 - protein, 123 aa, Homo sapiens
  Heavy chain of antibody 3T0478
VQLLQESGPGLVKPSETLSLTCTVSGGSISSYYWNWIRQSPGKGLEWMGYIYYTGSTNYNPSLKSRVTISVDLSKNQFSLKLRSVTAADTAVYYCARDKRYYDSSGYSHFDYWGQGILVTVSS

SEQ ID NO: 60 - protein, 110 aa, Homo sapiens
  Light chain of antibody 3T0478
QSVLTQPPSASGTPGQRVTIFCSGSSFNIGSNYVYWYQQLPGTAPKLLIYRNNERPSGVPDRFSGSKSGTSASLDISGLRSEDEADYSCAAWDGSLSGPVFGGGTKLTVL

SEQ ID NO: 61 - protein, 127 aa, Homo sapiens
  Heavy chain of antibody 3T0553
QVQLVESGGGVVQPGRSLRLSCAASGFTFTTCAMHWVRQAPGKGLEWVSVMWYDGSREYYADSVKGRFTISRDNSKNTLYLEMQSLRAEDTAVYYCARGGDLLVEVDSTDYYGLDVWGQGTTVTVSS

SEQ ID NO: 62 - protein, 107 aa, Homo sapiens
  Light chain of antibody 3T0553
DIQMTQSPSAMSASVGDRVTITCRASQGISHSLAWFQQKPGKVPKRLIYSASTLQSGVPSRFSGSGSGTEFTLTISSLQPEDFATYYCLQHNRYPWTFGQGTKVEIK

SEQ ID NO: 63 - protein, 121 aa, Homo sapiens
  Heavy chain of antibody 3T0611
EVQLVESGGNLVQPGGSLRLSCAASGFTFRSYSMNWVRQAPGKGLEWVSYISSSASIIHYAESVQGRFTISRDNAKNSLFLQMNSLRAEDTAVYYCAGILDDYRDYGYDYWGQGTLVTVSS

SEQ ID NO: 64 - protein, 109 aa, Homo sapiens
  Light chain of antibody 3T0611
SYELTQPPSVSVSPGQTARITCSGDALPKQYAYWYQQRPGQAPVLVIYKDIERPSGIPERYSGSSSGAIVTLTISGAQAEDEADYYCQSSDNNGSYRVMFGGGTKLTVL

SEQ ID NO: 65 - protein, 119 aa, Homo sapiens
  Heavy chain of antibody 3T0650
EVQLVQSGGGLVKPGGSLRLSCAASGFTFSNAWMNWVRQAPGKGLEWIGRVKSQTDRGTKDYAAPVKGRFSISRDDSKKMLYLQMNSLKIEDTGVYYCVATPHSWSVAWGQGTLVTVSS

SEQ ID NO: 66 - protein, 112 aa, Homo sapiens
  Light chain of antibody 3T0650
QSVLTQPPSVSGAPGQRVTISCTGSSSNIGAGYDVQWYQQFPGTAPKLLIYGNINRPSGVPDRFSGSKSGSSASLAITRLQAEDEADYYCQSYDSSLRDQWVFGGGTKLTVL

SEQ ID NO: 67 - protein, 123 aa, Homo sapiens
  Heavy chain of antibody 3T0673
EVQLVESGGGRVKPGGSLRVSCAVSGITFTNAWMTWVRQAPGKGLEWLGRIKSRSDGGQSDLIAPVKGRFTISRDDSKNILYLQMNNLKTEDTGLYFCTTYYYDNGGSYSNPWGLGTLVTVSS

SEQ ID NO: 68 - protein, 112 aa, Homo sapiens
  Light chain of antibody 3T0673
QSVLTQPPSVSGAPGQRVTISCTGSYSNIGAGYDVLWYQQLPGTAPKLLIYGNNKRPSGVPDRFSGSKSGTSASLAITGLQAEDEADYYCQSYDSRLRDFGIFGGGTKLTVL

SEQ ID NO: 69 - protein, 120 aa, Homo sapiens
  Heavy chain of antibody 4m0333
EVQLLESGGGLIQPGGSLRLSCAASGFAFSSYAMNWVRQAPGKGLEWVSGITGSGGTTYYPDSMKGRFTISRDNSKNTLYLQMNSLGGEDTATYYCAKGDDFWSGSPDVWGQGTTVTVSS

SEQ ID NO: 70 - protein, 111 aa, Homo sapiens
  Light chain of antibody 4m0333
QSVLTQPPSVSAAPGQKVTISCSGSSSNIGTNYVSWYQQLPGAAPKLLIYDNNKRPSGIPDRFSGSKSGTSATLGITGLQTGDEADYYCGTWDSSLSTFYVFGGGTKVNVL

SEQ ID NO: 71 - protein, 116 aa, Homo sapiens
  Heavy chain of antibody 4m0368
QVQLQESGPGLVKPSGTLSLTCAVSGGSISGTNWWTWVRQPPGRGLEWIGEIYYRGSPNYNPSLKSRVTISVDQSKSQFSLKLTSVTAADTAVYYCLRLSHYGGAWGQGTLVTVSS

SEQ ID NO: 72 - protein, 107 aa, Homo sapiens
  Light chain of antibody 4m0368
DIQLTQSPSSLSASVGDRVTITCRASQSISTYLNWYQQKPGKAPKFLIFSASNLQSGVPSRFSGGGSGTDFTLTISSLQPEDFATYYCQQTYNTPRTFGQGTKVEIK

SEQ ID NO: 73 - protein, 122 aa, Homo sapiens
  Heavy chain of antibody 4m0373
QVQLVESGGGLVKPGGSLRLSCAASGFTFSDHYMGWIRQAPGKGLEWVSYISTYATTAYYADSVKGRFTISRDNAKNSLYLQMNSLRAEDTAVYYCARGERRHQVLSGMDVWGQGTTVTVSS

SEQ ID NO: 74 - protein, 105 aa, Homo sapiens
  Light chain of antibody 4m0373
FMTQAPSSLSVSPGERATLSCRASQSVSSNLAWYQQKPGQAPRLLIYGASTRATGIPARFSGSGSGTEFTLTISSLQSEDFGVYYCQQCNTWPLAFGGGTKVEIK

SEQ ID NO: 75 - protein, 124 aa, Homo sapiens
  Heavy chain of antibody 4T0115
EVQLVESGGGLVQPGRSLRLSCVASGFTFDNYAMHWVRQIPGKGLEWVSTITWNSGRTGYADSVKGRFTISRDNAKNSLYLQMNTLRAEDTALYYCAREGRDGYNLPYYAMDVWGQGTTVTVSS

SEQ ID NO: 76 - protein, 108 aa, Homo sapiens
  Light chain of antibody 4T0115
DIQLTQSPSSLSASIGDRVTITCRASQGISSYLAWYQQKPGKAPKLLIYAASTLQSGVPSRFSGSGSGTDFTLTISSLQPEDFATYYCQRFNSYPIFTFAPGTKVDIK

SEQ ID NO: 77 - protein, 117 aa, Homo sapiens
  Heavy chain of antibody 4T0154
QVQLVQSGAEVKKPGSSVKVSCKTSGGNFGRFSISWVRQAPGQGLEWMGRIIPTLNMRNFAQRFQGRVTITADKSTSTAYMEVTSPTFEDSAVYYCAVEMATPSDTWGQGTLVTVSS

SEQ ID NO: 78 - protein, 107 aa, Homo sapiens
  Light chain of antibody 4T0154
DIQLTQSPSSLSASVGDRVTITCRASQGISNFVAWFQQKPGKVPKSLIYAGSRLQSGVPSKFSGSGSGTDFTLTISSLQPEDFATYYCLQYSTYPYTFGQGTKLEIK

SEQ ID NO: 79 - protein, 114 aa, Homo sapiens
  Heavy chain of antibody 4T0159
QLQLQESGPGLVKPSETLSLTCTVSGGSISTSSHYWGWIRQPPGKGLEWVGTRSNIGNTYYNPSLKSRVTIFVDTSKNQFSLRLSSVTAADTAVYYCARRFDSWGQGILVTVSS

SEQ ID NO: 80 - protein, 107 aa, Homo sapiens
  Light chain of antibody 4T0159
EIVMTQSPATLSVSPGERATLSCRASQSVSSNLAWFQQKPGQAPRLLIYGASTGVTGIPAKFSGSGSGTEFTLTIGSLQSEDFAVYYCQQYNDWPWTFGQGTKVEIK

SEQ ID NO: 81 - protein, 114 aa, Homo sapiens
  Heavy chain of antibody 4T0176
QLQLQESGPGLVKPSETLSLTCTVSGGSISSSSYYWGWIRQPPGKGLEWIGTISHSGSTYYNPSLQSRVTMSVDTSKNHFSLRLSSVTAADTAVYYCVRRFDYWGQGTLVTVSS

SEQ ID NO: 82 - protein, 107 aa, Homo sapiens
  Light chain of antibody 4T0176
EIVMTQSPATLSVSPGERATLSCRASQSVTSNLAWFQQQPGQAPRLLIYGASTRATGIPARFSGSGSGTEFTLTISSLQSEDLAVYYCQQYNKWPWTFGQGTKVEIK

SEQ ID NO: 83 - protein, 127 aa, Homo sapiens
  Heavy chain of antibody 4T0182
QVQLVQSGAEVKKPGASIKVSCKASGYIFTDYYMHWVRQAPGQGLEWMGWLNPKSGGTNYAQKFQGRVTMTRDTSINTAYMELNSVRSDDTAVYYCAAPGETIVRGVIYHYYGMGVWGQGTAVTVSS

SEQ ID NO: 84 - protein, 111 aa, Homo sapiens
  Light chain of antibody 4T0182
DIVMTQSPLSLPVTPGEPASISCRSSQSLLHRNGYNYLDWYLQKPGQSPQLLIYMGSNRASGVPDRFSGSGSGTDFTLKISRVEAEDVGVYYCMQALQTPTFGQGTKLEIK

SEQ ID NO: 85 - protein, 125 aa, Homo sapiens
  Heavy chain of antibody 4T0243
QVQLQESGPGLVKPSETLSLTCTVSGGSLSTYYWNWIRQPPGKGLEWIGYIYYSSLPKYSPTLKSRITISVDTSKNQFSLKLSSVTAADTAVYYCARAPRGYSSGKYNYYGLDVWSQGTTVTVSS

SEQ ID NO: 86 - protein, 108 aa, Homo sapiens
  Light chain of antibody 4T0243
SYELTQPPSVSVSPGHTATITCSGDALSKQFAYWYQQKPGQAPVLVISKDTERPSGIPGRFSGSSSGPTATLTISGVQAEDEADYYCQSADSSGGYYVFGSGTRVTVL

SEQ ID NO: 87 - protein, 121 aa, Homo sapiens
  Heavy chain of antibody 4T0262
QVQLVQSGAEVKKPGASVKIACKASGYTFRNYYMNWVRQAPGKGLEWVGIISPRGGTTRYAPKFQGRVTMTSDTSTTTVFMELNSLRSEDTAMYYCARGITMIRDGWFDHWGQGTLVTVSS

SEQ ID NO: 88 - protein, 108 aa, Homo sapiens
  Light chain of antibody 4T0262
DIQMTQSPSTLSASVGDRVTITCRASQSISNWLAWYQQKPGKAPKLLIYDASNLESGVPSRFSGSGSGTDFTLIIKSLQPDDFGSYFCLQYSSSSPYSFGQGTKLEIK

SEQ ID NO: 89 - protein, 122 aa, Homo sapiens
  Heavy chain of antibody 4T0284
QVQLVQSGAEVKKPGASVKVSCKASGYTFTKYGISWLRQAPGQGLEWMGWISTYNGDTNYAQKVQGRVTMTTDTSSTTAYMELRSLRSDDTAVYFCARGITMVRGLDWFDPWGQGTLVTVSS

SEQ ID NO: 90 - protein, 107 aa, Homo sapiens
  Light chain of antibody 4T0284
EIVMTQSPATLSVSPGERATLSCRASQNISSNLAWYQQKPGRAPRLLIDGASTRATGIPARFSGSGSGTEFTLSISSLQSEDSTVYYCQQYKNWPLTFGGGTKVEIK

SEQ ID NO: 91 - protein, 129 aa, Homo sapiens
  Heavy chain of antibody 4T0306
QVQLQQSGPGLVRPSQTLSLICAISGDSVSDNNAAWNWIRQSPSRGLEWLGRTYFRSRWYNDYPESLKGRVIINPDTAENQVSLQLSSVTPGDTAVYYCVREYYDLWNGFYPGGAMDVWGQGTTVTVSS

SEQ ID NO: 92 - protein, 107 aa, Homo sapiens
  Light chain of antibody 4T0306
DIQLTQSPSSLSASVGDRVTITCRASQDISDFLNWFQQKPGKAPKLLIYDVSKLARGVPSRFSGSASGTDFTFTISSLQPEDIATYYCQQYDDLPRTFGQGTKLEIK

SEQ ID NO: 93 - protein, 125 aa, Homo sapiens
  Heavy chain of antibody 4T0344
EVQLLESGGGLVQPGGSLRLSCATSGFTFSSYAMNWVRQAPGKGLEWVSSIGDTGGSKYYVDPVKGRFTVSRDNSKNTLYLQMNSLRAEDTAVYYCVKGSWPRTTVLSTYYFDYWGQGTLVTVSS

SEQ ID NO: 94 - protein, 107 aa, Homo sapiens
  Light chain of antibody 4T0344
DIQMTQSPSSLSASVGDRVTITCRASQGISNYLAWYQQKPGQSPKLLIYAASTLQFGVPTRFSGSGSGADFTLTISSLQPEDVATYYCQKYNGAPWRFGQGTKVEIK

SEQ ID NO: 95 - protein, 122 aa, Homo sapiens
  Heavy chain of antibody 4T0365
QLQLQESGPGLVKSSETLSLTCTVSGDSIRSDSHYWDWIRQPPGKGLEWIGNIYYSGSTYYNPSLKSRVTISIDTSKSQFSLRLSSVTAADTALYYCATSSRGSVSYYSRYWGQGTLVTVSS

SEQ ID NO: 96 - protein, 110 aa, Homo sapiens
  Light chain of antibody 4T0365
QSVLTQPPSVSAAPGQKVTISCSGSSSNIGLNYVSWYQQFPGTAPKLLIYDNIQRPSGIPDRISGSKSGTSSTLAITGLQTGDEADYFCSVWDSSLGSRVFGGGTKLTVL

SEQ ID NO: 97 - protein, 126 aa, Homo sapiens
  Heavy chain of antibody 4T0412
QVQLVESGGGVVQPGRSLRLSCAASGFTFSRFGLHWVRQVPDKGLEWVALIWYDGNDRYYADSVKGRFTVSRDNSKNTLFLEMNSLRAEDTAVYYCVRETFQPGGEVPPLWYFDVWGRGTLVTVSS

SEQ ID NO: 98 - protein, 107 aa, Homo sapiens
  Light chain of antibody 4T0412
DIQLTQSPSSLSASVGDRVTITCRASQSITTNLNWYQQKPGKAPNLLIFAASSLQSGVPSRFSGSGSGTDFTLTITSLQPEDFATYYCQQSYGTPRTFGQGTKVEIK

SEQ ID NO: 99 - protein, 123 aa, Homo sapiens
  Heavy chain of antibody 4T0423
EVQLVESGGGWVQPGRSLRLSCVASGFTFQNYAMHWVRQPPGKGLEWVSGISWNSGSIGYADSVKGRFTISRDNARNSLYLQMRSLRPDDTALYYCAKENRIELLPGWGMDVWGQGTTVTVSS

SEQ ID NO: 100 - protein, 112 aa, Homo sapiens
  Light chain of antibody 4T0423
QSALTQPASVSGSPGQSITISCTGPSSDVWSYNLVSWYQQFPGKAPKLMIYEDTKRPSGVSIRFSGSKSGNTASLTISGLQAEDEADYFCCSYAGTSAFVWVFGGGTKLTVL

SEQ ID NO: 101 - protein, 121 aa, Homo sapiens
  Heavy chain of antibody 4T0427
EVQLVESGGDLIQPGGSLRLSCAASGFFVSSSYMSWVRQAPGKGLEWVSAIYSGGTTFYVDSVKGRFTISRDNSKNTLYLQMNSLRAEDTAVYYCARDEEWLFSGYGMDVWGQGTTVTVSS

SEQ ID NO: 102 - protein, 110 aa, Homo sapiens
  Light chain of antibody 4T0427
QSVLTQPPSTSGTPGQRVTISCSGSSSNIGLNYVYWYQQLPGTAPKLLIYRNNQRPSGVPDRFSGSKSGTSASLAISGLRSEDEADYYCAAWDDSLSAYIFGTGTKVTVL

SEQ ID NO: 103 - protein, 116 aa, Homo sapiens
  Heavy chain of antibody 4T0429
QVQLQQWGAGLLKPSETLSLTCAVYRGSFITYYWSWVRQPPGRGLEWIGEIYKDGSASYNPSLKNRVTISLDMSKNHFSLNLDSVTAADTAVYYCARHGHTGMDVWGQGTTVTVSS

SEQ ID NO: 104 - protein, 110 aa, Homo sapiens; identical to SEQ ID NO: 102
  Light chain of antibody 4T0429

SEQ ID NO: 105 - protein, 121 aa, Homo sapiens
  Heavy chain of antibody 4T0434
EVQLVESGGGLIQPGGSLRLSCAASGFTLRNYWMHWVRQAPGKGLVWVSRINSDGSTTNYADSVKGRFTISRDNAKNTLYLQMRSLTAEDTAVYYCARDVGLYNTGWIDYWGQGAPVTVSS

SEQ ID NO: 106 - protein, 113 aa, Homo sapiens
  Light chain of antibody 4T0434
DIVMTQSPDSLAVSLGERATINCKSSQSVLHSSNNKYYLAWYQQKPGQPPKLLIYWASTRGSGVPDRFSGSGSGTDFTLTISSLQAEDVAVYYCQQFYSTPLTFGGGTKVEIK

SEQ ID NO: 107 - protein, 118 aa, Homo sapiens
  Heavy chain of antibody 4T0444
EVQLVVSGGGLVQPGGSLRLSCAASGFTLKSYDIHWVRQRPGEGLEWVAAIGAAGDTYFSGSVKGRFTISRENARNSLYLQMNSLGAGDTAIYYCARARFGDLALDYWGQGILVTVSS

SEQ ID NO: 108 - protein, 108 aa, Homo sapiens
  Light chain of antibody 4T0444
EIVLTQSPGTLSLSPGERATLSCRSSQSVSSNSLAWYQQKPGQAPRLLIYGASSRAAGIPDRFSGSGSGTDFTLTISRLQPEDFAVFFCHQYSSSPLTFGGGTKVEIK

SEQ ID NO: 109 - protein, 119 aa, Homo sapiens
  Heavy chain of antibody 4T0452
EVQLVESGGGLVKPGGSLRISCAASGFSFSNAWMNWVRQAPGKGLEWVGRIKSVSDGGPIHYAAPVKGRFTISRDDSKKMLYLQMNSLKVDDTAVYYCATEPRGYDWGWGQGTLVTVSS

SEQ ID NO: 110 - protein, 112 aa, Homo sapiens
  Light chain of antibody 4T0452
QSVLTQPPSVSGAPGQRVTISCTGSYSNIGAGYDVYWYQQFPGTAPKLLIYGNTNRPSGVPDRFSGSRSGTSASLAITGLQAEDEAHYYCQSYDSRLRDNWVLGGGTKLTVL

SEQ ID NO: 111 - protein, 117 aa, Homo sapiens
  Heavy chain of antibody 4T0525
QVQLVQSGPEVKKPGSSVKVSCKASGGRFSTSTFNWVRQAPGQGLEWMGRIIPIIATTDYAQKFQGRVTITADKSTGTAYMELSRLRSDDTAVYYCAGGGTGLFDSWGQGTLVTVSS

SEQ ID NO: 112 - protein, 107 aa, Homo sapiens
  Light chain of antibody 4T0525
DIQMTQSPSSLSASVGDRVTITCRASQGISNYLAWFQQKPGEAPKSLIYEASSLQSGVPSKFSGSGSGTDFSLTISSLQPEDFATYYCQQYNTLPFTFGGGTKVEIK

SEQ ID NO: 113 - protein, 123 aa, Homo sapiens
  Heavy chain of antibody 4T0541
EVQLLESGGGLEEPGGSLRLSCTVSGFSFSMYTMTWVRQVPGKGLEWVSAVSGTSGGTYYADSVKGRFTISRDNSKNTLYLQMNSLRAEDTAVYYCAKVSDYLWGSHRYFDYWGQGTLVTVSS

SEQ ID NO: 114 - protein, 107 aa, Homo sapiens
  Light chain of antibody 4T0541
DIQMTQSPSTLSASVGDRVTVTCRASHSVSGRLAWYQQKPGKAPKLLISDASSLESGVPSRFSGSGSGTEFTLTISSLQPDDFATYFCQQYNSYSPTFGQGTKVEIK

SEQ ID NO: 115 - protein, 124 aa, Homo sapiens
  Heavy chain of antibody 4T0570
QVQLVQSGAEVKKPGASIKVSCKASGYTFVSKYIHWVRQAPGQGLEWMGIINPSGGRTDYAQKFQGRVTISRDTSTSTVYMELSSLRSEDTAFYYCARALPFRVTGARDVFDIWGQGTMVTVSS

SEQ ID NO: 116 - protein, 107 aa, Homo sapiens
  Light chain of antibody 4T0570
DIQMTQSPSSLSASVGDRVTITCRASQGISTNLAWFQQRPGKAPKSLVYAASTLQNGVPSKFSGSGSGTDFTLTISSLQPEDFATYYCQQYYTYPLTFGGGTKVEIK

SEQ ID NO: 117 - protein, 118 aa, Homo sapiens
  Heavy chain of antibody 4T0578
EVQLVESGGALVQPGGSLRLSCAASGFTFSRYDIHWVRQASGKGLEWVSAIGKAGDAYYAGSVRGRFTISRENAKNSLYLQMNSLRAGDTAVYYCARASFGDLYFDLWGRGTLVTVSS

SEQ ID NO: 118 - protein, 113 aa, Homo sapiens
  Light chain of antibody 4T0578
DIVMTQSPDSLAVSLGERATINCKSSQSLLHSSNNKNSVAWYQQKPGQPPKLLIYWASTREFGVPDRFSGSGSGTDFALTISSLQAEDVAVYVCQQYYNSPLTFGPGTKVDIK

SEQ ID NO: 119 - protein, 121 aa, Homo sapiens
  Heavy chain of antibody 4T0657
EVQLLESGGDLVQPGGSLRLSCTASGFTFKDFAMNWVRQAPGKGLEWVSGITGSGGTTYYADSVKGRFTSSRDNSKNTLDLQMNSLGVDDTAVYYCAKNDDFWGGPTFDYWGQGTLVTVSS

SEQ ID NO: 120 - protein, 109 aa, Homo sapiens
  Light chain of antibody 4T0657
QSVLTQPPSVSAAPGQKVTISCSGISSNIGNNYVSWYQQLPGTAPKLLIYDNNKRPSGIPDRFSASKSGTSATLGITGLQTGDEADYYCGTWDITLSEGVFGGGTKLTV

SEQ ID NO: 121 - protein, 121 aa, Homo sapiens
  Heavy chain of antibody 4T0726
QVQLVQSGVEVKEPGASVRVSCKASGYTFTHYGITWVRQAPGQGLEWMGWISGYNGNTDYAQKFQGRITMTTDTSTSTAYMELGRLTSDDTAVYYCARTITMILLGWFDPWGQGTLVTVSS

SEQ ID NO: 122 - protein, 108 aa, Homo sapiens
  Light chain of antibody 4T0726
EIVLTQSPGTLSLSPGERATLSCRASQSVSSNYLAWYQQKPGQAPRLLISGASNRATGLPDGFSGSGSGTDFTLTISRLEPEDFAVFFCQQYGTSPLTFGPGTKVDIK

SEQ ID NO: 123 - protein, 116 aa, Homo sapiens
  Heavy chain of antibody 4T0764
QVQLQESGPGLVEPSETLSLTCTVSGASISSLFWSWIRQPPGKGLEWIGYIHSDGSTNYNPFLKSRVTISIDTSQNQFSLRLTSVTAADTAVYFCARGLLGSQDSWGQGTLVTVSS

SEQ ID NO: 124 - protein, 111 aa, Homo sapiens
  Light chain of antibody 4T0764
QSALTQPASVSGSPGQSITISCTGTSSDVGGYNYVSWYQQYPGKAPKLVIYEVTNRPSGVSNRFSGSKSGNTASLTISGLRAEDEADYHCSSYRTRNTHVIFGGGTKLTVL

SEQ ID NO: 125 - protein, 127 aa, Homo sapiens
  Heavy chain of antibody 4T0784
QVQLVESGGGVVQPGRSLRLSCAASGFTFSTYGVHWVRQAPGKGLEWVGVIWYDGSKKYYADSVKGRFTISRDNSKNTLYLQMNSLRAEDAAVYRCVRGGDLLLLPDSIDYYGMDVWGQGTLVTVSS

SEQ ID NO: 126 - protein, 107 aa, Homo sapiens
  Light chain of antibody 4T0784
KDHDPQAPVSLSASVGDRVTITCRASQGIRNDLGWYQQKPGKAPKRLIYAASSLQSGVPSRFSGSGSGTEFTLTISSLQPEDFATYYCLQHNTYPFSFGPGTKVDIK

SEQ ID NO: 127 - protein, 124 aa, Homo sapiens
  Heavy chain of antibody 5T0107
QVQLVESGGTVVQPGRSLRLSCAASGFMFSDYGMNWVRQAPGKGLEWVNFISSDGTRTYFADSVKGRLTISRDNSKNMVFLEMNSLRPEDTAIYYCAKDRYCDAGTCSGSLDYWGQGTLVTVSS

SEQ ID NO: 128 - protein, 112 aa, Homo sapiens
  Light chain of antibody 5T0107
DIVMTQSPLYLPVTPGEPASISCRSNQSLLHSNGNKYLDWYLQKPGQSPQLLIYLGSTRASGVSDRFSGSGSDTDFTLKISRVEADDVGVYYCMQSLAARPTFGQGTKVEIK

SEQ ID NO: 129 - protein, 124 aa, Homo sapiens
  Heavy chain of antibody 5T0180
EVQLVESGGGLVKPGGSLRLSCAASGFSFGNAWMNWVRQAPGKGLEWVGRIKSKSDGGTTDYAAPVKDRFIISRDDSKKTLYLQMNSLRREDTAVYYCVRGPFYCDTCGPNDYWGQGTLVTVSS

SEQ ID NO: 130 - protein, 112 aa, Homo sapiens
  Light chain of antibody 5T0180
QSVLTQPPSVSGAPGQRVTISCTGSSSNIGAGYDVQWYQQLPGTAPKVLIYGNNNRPSGVPDRFSGSKSGSSASLAITGLQAEDEADYYCQTYDSRLRDQWVFGGGTKLTVL

SEQ ID NO: 131 - protein, 122 aa, Homo sapiens
  Heavy chain of antibody 5T0202
EVQLVESGGGLVKPGGSLRLSCGASGFSFNDAWMNWVRQAPGKGLEWVGRIKSKADGGGSDTAAPVKGRFIISRDDSKNTLFLQMERLKSEDTGVYYCTTSLNIWGTYRDCWGQGTLVTVSS

SEQ ID NO: 132 - protein, 112 aa, Homo sapiens
  Light chain of antibody 5T0202
QSVLTQPPSVSGAPGQRVTISCTGSYSNIGAGYDVRWYQQLPETAPKLLIYGNNNRPSGVPDRFSGSKSGTSASLAITGLQAEDEADYYCQSYDSRLRDSWVFGGGTRLTVL

SEQ ID NO: 133 - protein, 119 aa, Homo sapiens
  Heavy chain of antibody 5T0209
EVQLVDSGGGLVKPGGSLRLSCAASGFTFSKAWMSWVRQAPGKGLEWVGRIKSRTDGGTTDYAAPVRGRFTISRDDSKNTLFLQMNSLKPEDTAIYYCATDYYESRGYWGQGTLVTVSS

SEQ ID NO: 134 - protein, 112 aa, Homo sapiens
  Light chain of antibody 5T0209
QSVLTQPPSVSGAPGQRVTISCTGSSSNIGAGYDVNWYQQIPGTAPKLLIYGNNKRPSGVPDRFSGSKSGTSVSLAITGLQAEDEADYYCQSYDSRLSDHWVFGGGTKLTVL

SEQ ID NO: 135 - protein, 118 aa, Homo sapiens
  Heavy chain of antibody 5T0223
EVQLVESGGGLVKPGGSLRLSCEASGFTFRNTWMNWVRQAPGKGLEWVGRIKSNTDGGTADYAAPVKGRFTISRDDSKSTLFLEMISLRTEDTGFYYCTKGGPGAHSWGLGTLVTVSS

SEQ ID NO: 136 - protein, 112 aa, Homo sapiens
  Light chain of antibody 5T0223
QSVLTQPPSVSGAPGQRVTISCTGSSSNIGAGYDVQWYQQLPGTAPKVLIYHNTNRPSGVPDRFSGSRSGTSASLVITGLQAEDEADYYCQTYDSRLSDHWVFGGGTKLTVL

SEQ ID NO: 137 - protein, 121 aa, Homo sapiens
  Heavy chain of antibody 5T0257
EVQLLESGGGLVQPGGSLRLSCAASGFTFSSYAMNWVRQAPGKGLEWVSAISGSGGSTYYADSVKGRFSISRDNSKNTLYLQMNSLRAEDTAVYYCAKDDDFWSGPYFDYWGQGTLVTVSS

SEQ ID NO: 138 - protein, 110 aa, Homo sapiens
  Light chain of antibody 5T0257
QSVLTQPPSVSAAPGQKVTISCSGSSSNIGYNYVSWYQQLPGTAPKLLIYENNKRPSGIPDRFSGSKSGTSATLGITGLQTGDEADYYCGSWDSSLSTGVFGGGTKLTVL

SEQ ID NO: 139 - protein, 120 aa, Homo sapiens
  Heavy chain of antibody 5T0278
EVQLVESGGGLVKPGGSLRVSCAASGFPFTDAWMSWVRQAPGKGLEWVGRIKSNSDGGIPDYAAPVKGRFTISRDDSKRTLYLQMNSLKTEDTGYYYCYTLRFFGPGNSWGQGTLVTVSS

SEQ ID NO: 140 - protein, 112 aa, Homo sapiens
  Light chain of antibody 5T0278
QSVLTQPPSVSGAPGQRITISCTGSYSNIGAGYDVSWHQQLPGKAPKLLIYGNNNRPSGVPDRFSGSKSGTSASLTITGLQAEDEADYYCQSYDARLRDQVVFGGGTKLTVL

SEQ ID NO: 141 - protein, 122 aa, Homo sapiens
  Heavy chain of antibody 5T0337
EVQLVESGGGLVKPGGSLRISCAASGFTFNNAWMNWVRQSPGKGLEWVGRIKSKSDAGTTDYAAPVKGRFIISRDDSKKTLFLQMNSLKIEDTAIYFCMTGFEHTYSYFDLWGRGTQVTVSS

SEQ ID NO: 142 - protein, 113 aa, Homo sapiens
  Light chain of antibody 5T0337
QSVLTQPPSVSGAPGQRVIISCIGGSSNIGAGYDVKWYQQLLPGTAPRLLIYGNTNRPSGVPDRFSGSKSGTSASLAITGLQAEDEADYYCQSYDSRLRDAWVFGGGTKLTVL

SEQ ID NO: 143 - protein, 116 aa, Homo sapiens
  Heavy chain of antibody 5T0376
QVQLQESGPGLVKPSETLSLTCSVSGASISTLYWSWIRQPPGKGLEWIGYMHSSGTTNYNPSLESRVTMSIDTSRNQFSLKLTSVTAADTAVYYCARGLLGPGDSWGQGTLVTVSS

SEQ ID NO: 144 - protein, 111 aa, Homo sapiens
  Light chain of antibody 5T0376
QSALTQPASVSGSPGQSITISCTGTSSDVGGYNYVSWYQQYPGKAPKLMIYEVTSRPSGVSNRFSGSKSGNTASLTISGLQAEDEGDYYCSSYKTGSTPVVFGGGTKLTVL

SEQ ID NO: 145 - protein, 114 aa, Homo sapiens
  Heavy chain of antibody 5T0378
QLQLQESGPGLVKPSETLSLTCTVSGGSISSSSYYWGWIRQPPGKGLEWIGTISYSGSTYYNPSLKSRVTISLDTSKNQFSLRLSSVTAADTAVYYCARRFDYWGQGTLVTVSS

SEQ ID NO: 146 - protein, 107 aa, Homo sapiens
  Light chain of antibody 5T0378
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIK

SEQ ID NO: 147 - protein, 125 aa, Homo sapiens
  Heavy chain of antibody 5T0404
QVQLVQSGAEVKKPGSSVKLACKASGDTFTTYPITWVRQAPGRGLEWMGRIIPMLAAETYAQKFLGRLAITADKSTNTVYMVLNSLRSEDTAVYFCARLYCSSSSCSIYNYMDVWGKGTTVTVSS

SEQ ID NO: 148 - protein, 107 aa, Homo sapiens
  Light chain of antibody 5T0404
DIQMTQSPSSLSASVGDRVTITCQASQDISNYLNWYQQKPAKAPELLIYDASNLEAGVPSRFSGGGSGTDFSLTINSLQPEDIATYYCQQYDSLPYSFGQGTKLEIK

SEQ ID NO: 149 - protein, 126 aa, Homo sapiens
  Heavy chain of antibody 5T0406
QVQLVQSGPEVKKPGSSVRVSCKPSGGTFNSYAITWVRQAPGQGLEWMGGIIPIFDTPKYAQKFQDRVTLTADESTTTTYMELNSLRSEDTAVYFCARFRFFQGGGSFYYYGLDLWGQGTAVTVSS

SEQ ID NO: 150 - protein, 112 aa, Homo sapiens
  Light chain of antibody 5T0406
QSVLTQPPSASGTPGQRVTISCSGSNSNIGSNPVNWYQHLPGTAPKLLIYSDNQRPSGVPDRFSGSKSGTSASLAISGLQSEDEADYYCAAWDDRLNGPGYVFGTGTTVTVL

SEQ ID NO: 151 - protein, 114 aa, Homo sapiens
  Heavy chain of antibody 5T0420
QLQLQESGPGLVKPSETLSLVCTVSGGSISSSSFYWGWIRQPPGKGLEWIATISNLGSTYYNPSLKSRVTISVDTSRKHLSLKLNSVTAADTAVYFCARRFDLWGRGTLVTVSS

SEQ ID NO: 152 - protein, 107 aa, Homo sapiens
  Light chain of antibody 5T0420
EIVMTQSPATLSVSPGQRATLSCRASQSVGTNLAWFQQKPGQAPRLLIYGESTRATDIPARFSGSGSGTDFTLTITSLQSEDFAVYYCQQYNIWPWTFGQGTKVEIK

SEQ ID NO: 153 - protein, 123 aa, Homo sapiens
  Heavy chain of antibody 5T0451
EVQLVESGGGLVKPGGSLRLSCAASGFTFTNAWMNWVRQAPGKGLEWIGRIKSKGDGGTADHAAPVRGRFIISRDDSKQMLYLQMTGLKSDDTAVYYCTRGPYDNFSGWRDYWGQGTLVTVSS

SEQ ID NO: 154 - protein, 112 aa, Homo sapiens
  Light chain of antibody 5T0451
QSVLTQPPSVSGAPGQRVTISCTGSGSNIGAGYDVQWYQQHPGTAPKVLIYGNNKRPSGVPDRFSGSKSGTSASLAITGLQAEDEAHYYCQSYDARLRDSWVFGGGTQLTVL

SEQ ID NO: 155 - protein, 119 aa, Homo sapiens
  Heavy chain of antibody 5T0465
QVQLVQSGAEVREPGASVRISCKASGYTFTSASIHWVRQAPGQGLEWMGIIDPVVGKTRSERKFNGRLIMTRDTPTSTVYMELNSLRSDDTAMYYCARSGYYPRGMDVWGQGTTVTVSS

SEQ ID NO: 156 - protein, 107 aa, Homo sapiens
  Light chain of antibody 5T0465
SYELTQPPSVSVSPGQTASITCSGDKLGDEYVSWYQQKPGQSPVLVISRDTRRSSGIPERFSGSNSGNTATLTISGTQAMDEADYYCQAWDSSTAVVFGGGTKLTVL

SEQ ID NO: 157 - DNA, 2142 bp, Artificial Sequence
  Nucleotide sequence of EBOV delta TM
atgggtgttacaggaatattgcagttacctcgtgatcgattcaagaggacatcattctttctttgggtaattatccttttccaaagaacattttccatcccgcttggagttatccacaatagtacattacaggttagtgatgtcgacaaactagtttgtcgtgacaaactgtcatccacaaatcaattgagatcagttggactgaatctcgaggggaatggagtggcaactgacgtgccatctgcgactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccatgtgccggagactttgccttccacaaagagggtgctttcttcctgtatgatcgacttgcttccacagttatctaccgaggaacgactttcgctgaaggtgtcgttgcatttctgatactgccccaagctaagaaggacttcttcagctcacaccccttgagagagccggtcaatgcaacggaggacccgtcgagtggctattattctaccacaattagatatcaggctaccggttttggaactaatgagacagagtacttgttcgaggttgacaatttgacctacgtccaacttgaatcaagattcacaccacagtttctgctccagctgaatgagacaatatatgcaagtgggaagaggagcaacaccacgggaaaactaatttggaaggtcaaccccgaaattgatacaacaatcggggagtgggccttctgggaaactaaaaaaaacctcactagaaaaattcgcagtgaagagttgtctttcacagctgtatcaaacggacccaaaaacatcagtggtcagagtccggcgcgaacttcttccgacccagagaccaacacaacaaatgaagaccacaaaatcatggcttcagaaaattcctctgcaatggttcaagtgcacagtcaaggaaggaaagctgcagtgtcgcatctgacaacccttgccacaatctccacgagtcctcaacctcccacaaccaaaacaggtccggacaacagcacccataatacacccgtgtataaacttgacatctctgaggcaactcaagttggacaacatcaccgtagagcagacaacgacagcacagcctccgacactccccccgccacgaccgcagccggacccttaaaagcagagaacaccaacacgagtaagagcgctgactccctggacctcgccaccacgacaagcccccaaaactacagcgagactgctggcaacaacaacactcatcaccaagataccggagaagagagtgccagcagcgggaagctaggcttaattaccaatactattgctggagtagcaggactgatcacaggcgggagaaggactcgaagagaagtaattgtcaatgctcaacccaaatgcaaccccaatttacattactggactactcaggatgaaggtgctgcaatcggattggcctggataccatatttcgggccagcagccgaaggaatttacacagaggggctaatgcacaaccaagatggtttaatctgtgggttgaggcagctggccaacgaaacgactcaagctctccaactgttcctgagagccacaactgagctgcgaaccttttcaatcctcaaccgtaaggcaattgacttcctgctgcagcgatggggtggcacatgccacattttgggaccggactgctgtatcgaaccacatgattggaccaagaacataacagacaaaattgatcagattattcatgattttgttgataaaacccttccggaccagggggacaatgacaattggtggacaggatggagacaaatgaaacagatagaggacaagatagaagagatcttaagcaaaatctaccatattgaaaatgagatcgcacggataaaaaaactgatcggtgaggtcgcatcaagctccatcgaggggcggggcagccaccaccatcatcaccacggtctgaacgacatcttcgaggctcagaaaatcgaatggcacgaatag

SEQ ID NO: 158 - protein, 713 aa, Artificial Sequence
  Protein sequence of EBOV delta TM
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYASGKRSNTTGKLIWKVNPEIDTTIGEWAFWETKKNLTRKIRSEELSFTAVSNGPKNISGQSPARTSSDPETNTTNEDHKIMASENSSAMVQVHSQGRKAAVSHLTTLATISTSPQPPTTKTGPDNSTHNTPVYKLDISEATQVGQHHRRADNDSTASDTPPATTAAGPLKAENTNTSKSADSLDLATTTSPQNYSETAGNNNTHHQDTGEESASSGKLGLITNTIAGVAGLITGGRRTRREVIVNAQPKCNPNLHYWTTQDEGAAIGLAWIPYFGPAAEGIYTEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQMKQIEDKIEEILSKIYHIENEIARIKKLIGEVASSSIEGRGSHHHHHHGLNDIFEAQKIEWHE